(19) United States
(12) Patent Application Publication
(10) Pub. No.: US 2007/0036839 A1
(43) Pub. Date: Feb. 15, 2007

(54) Phosphorylated glucomannane polysaccharides containing 1-6 and 1-2 linkages increase weight gain in poultry

(76) Inventors: Jose Antonio Matji Tuduri, Madrid (ES); Antonio F. Guerrero Gomez-Pamo, Madrid (ES); Jose Luis Alonso Lebrero, Madrid (ES); Garrett Lindemann, Sheridan, WY (US)

Correspondence Address: LATHROP & GAGE LC, 4845 PEARL EAST CIRCLE, SUITE 300, BOULDER, CO, 80301, US

(21) Appl. No.: 11/490,566

(22) Filed: Jul. 20, 2006

Related U.S. Application Data
(60) Provisional application No. 60/702,887, filed on Jul. 27, 2005.
(60) Provisional application No. 60/703,028, filed on Jul. 27, 2005.
(60) Provisional application No. 60/702,886, filed on Jul. 27, 2005.
(60) Provisional application No. 60/702,878, filed on Jul. 27, 2005.
(60) Provisional application No. 60/702,885, filed on Jul. 27, 2005.

(51) Int. Cl. A61K 31/715; A23K 1/165

(52) U.S. Cl. 424/442; 514/54

(57) ABSTRACT

Phosphorylated glucomannans may be purified from naturally occurring sources and used as a supplement to poultry feeds for the benefit of poultry production.

### RELATED APPLICATIONS

This application claims benefit of priority to provisional application Ser. No. 60/702,887 filed Jul. 27, 2005, provisional application Ser. No. 60/703,028 filed Jul. 27, 2005, provisional application Ser. No. 60/702,886 filed Jul. 27, 2005, provisional application Ser. No. 60/702,878 filed Jul. 27, 2005, and provisional application Ser. No. 60/702,885 filed Jul. 27, 2005.

### BACKGROUND

1. Field of the Invention

The present disclosure pertains to the supplementation of poultry diet with phosphorylated glucomannan polysaccharides to the benefit of poultry production. Particularly preferred advantages are increased rate of poultry weight gain, more efficient feed-to-gain and increased size of the poultry breast meat.

2. Description of the Related Art

Antibiotics may be added to the nursery, grower and finisher feeds of poultry to promote growth and/or reduce disease occurrence during all phases of food production. The purpose for addition of the antibiotics is to promote growth during the starter, grower and finishing phase of poultry production¹. The antibiotics promote growth through the reduction of biological stress, the decrease of malicious bacteria, and by promoting the health of the poultry. Poultry that are healthy and disease free eat more food, and more effectively convert the food into muscle or meat. Typically, subtherapeutic levels of antibiotics increase growth rate about 15% and improve efficiency of feed conversion from 5% to 7%. On the other hand, poultry that are unhealthy or not disease free, are stressed. Relatively more of the ingested fed energy is utilized to reduce or remove the biological stress the animal is facing. Thus, the antibiotic supplementation of poultry diet is shown to have numerous benefits.

Despite these advantages, the practice of supplementing poultry diet with antibiotics is increasingly problematic. Sub-therapeutic doses of antibiotics are linked to the increased presence of antibiotic-resistant bacterial strains in humans, animals and in the environment^{2,3}. It is also possible for residual antibiotics to appear in food that is meant for human consumption. The United States Food and Drug Administration (USFDA) requires the antibiotic must be with drawn from the feed of the poultry at least two weeks prior to slaughter to prevent the antibiotics sequestered in the poultry from being ingested by humans.

The problems resulting from subtherapeutic antibiotic usage are of such growing significance that various other regulatory agencies have taken keen interest. In one example of a regulatory response, the European Union has recently mandated that antibiotics may not be used as growth promoters in feed animals⁴. Over the years, antibiotics have been slowly restricted, culminating with the complete banning of antibiotics in the European Union as growth promoters commencing Jan. 1, 2006.

The restriction or banning of antibiotic supplements to animal diets has direct cost in terms of economics and animal health. The commercial cost of producing meat and milk from animals has increased and the health of the animals in high density production facilities has decreased. ^{1,2}

One alternative to the use of antibiotics as growth promoters includes oligosaccharide products that are derived from yeast cell walls and are composed of sugars such as galactose, fructose, and mannose.¹ These small fragments of carbohydrates may selectively stimulate some of the gut flora of an animal. This stimulation alters the microbial balance, resulting in a benefit to the host animal.³ Additionally, the animal may not digest some of the small fragments of carbohydrates. As one example, mannan oligosaccharides are not digested by poultry, and pass through the animal functioning as a soluble fiber. One benefit of this type of soluble fiber is a cleansing effect by detaching pathogens from the animal's gut^{5,1,3}, thereby removing the pathogens from the animal's gastrointestinal tract.

Growth promotion in broilers, chickens and turkeys by mannan oligosaccharide has been investigated and demonstrated to be effective. Studies indicate that inclusion of a commercially available mannan oligosaccharide, Bio-Mos®, in broiler diets allows the broilers to perform similar to broilers fed the same diet containing antibiotics on the parameters of feed conversion, weight gain, parts yield, dressing percentage and mortality⁶. Turkeys fed a diet containing Bio-Mos® (0.10%) performed as well as did turkeys fed a control diet containing an antibiotic. Parameters measured for comparison between groups included; intestinal breaking strength, body weight, mortality, breast meat yield, and feed conversion⁷.

Another study concluded that turkeys fed a diet containing a concentration of mannan oligosaccharides out performed the control groups and led to the conclusion that mannan oligosaccharides may be used as an alternative to antibiotics as a growth promotant to improve turkey performance⁸. Weanling swine diets containing mannan oligosaccharides or phosphorylated mannan oligosaccharides have been demonstrated to have a growth promoting effect^{9,10,11}. Additional research has indicated that supplementing a dry cow's diet with mannan oligosaccharide enhances the cow's response to rotavirus and tends to enhance the transfer of those rotavirus antibodies to claves¹². Furthermore, feeding fructooligosaccharide, mannanoligosaccharide, oligofructose and Inulin have been demonstrated to protect mice¹³ from enteric and systemic pathogens and tumor inducers as well as increase the immune status and colonic health of dogs¹⁴.

One benefit of feeding mannan oligosaccharides to chickens is the growth promotion of bacteria that are beneficial to the host; namely and as an example, species of Bifidobacterium and Lactobacillus; while decreasing the colonization and growth of unbeneficial bacterial species to the host; namely and as an example species of Enterbacteriaceae, Enterococcus and Salmonella^{15,16.}

In general, oligosaccharides, specifically the mannan family of carbohydrates, have been demonstrated to be potent immunostimulants; activating macrophages, stimulating T-cells and blocking phagocytosis. The response is elicited through the binding of the mannan to receptors that are located on the macrophage external surface and intercellularly^{17,18}. Acemannan (ACM 1) is a β-(1-4)-acetylated mannan isolated from Aloe vera that has been used in wound healing and as an adjuvant in vaccination¹⁹. Delivery of a single low dose of ACM 1 to a chicken by intramuscular injection has been demonstrated to result in a systemic immuno-modulated activation of macrophages¹⁹.

One example of an immune enhancing glucomannan reported in U.S. Pat. No. 4,138,479 issued to Truscheit, et al., which teaches the use of a glucomannan protein that is purified from yeast cells. An extraction protocol contacts the yeast with equal parts of phenol and water. Three phases including solids, phenol and water are separated by centrifugation. The aqueous phase is concentrated by dialysis and then lyophilized. The resulting solid composition induces an immunopotentiating response and so are somewhat effective against neoplasms.

Other glucomannans from aloe have been reported to have an immunopotentiating function. U.S. Pat. No. 6,271,214 issued to Qiu et al. describes the concentration of β-1,4 glucomannan from aloe by a combination of hydrolysis and chromatography. The β-1,4 glucomannan is useful as an immunomodulating or immunostimulating composition, and may be administered topically or orally to treat radiation and chemically induced swelling of murine ear tissues.

A phosphorylated glucomannan, in combination with a seed coat protein that is commonly known as Immunoferon or AM3 has been demonstrated to stimulate haemolytic plaque-forming B lymphocytes²⁰ as well as enhancing the number and activity of peripheral blood monocytes and macrophages, and cytotoxic activities of NK cells in humans exhibiting indications of chronic bronchitis and mice of an elderly age²¹. Further, the ability of Immunoferon to restore natural killer (NK) cell phagocytic cells to normal activity has been verified in humans²².

Additionally, Immunoferon, not only activities and restores not monocyte and macrophage cell function, but it also functions to reduce inflammation and inflammatory pathway activators. Specifically, Immunoferon has been demonstrated to reduce proinflammatory molecules such as Tumour Necrosis Factor α(TNF-α)²³. In the case of lipopolysaccahride induced TNF-α, research demonstrated that treatment with Immunoferon resulted in regulation of TNF-α through increased production of TNF-α such as Interleukin 10 (IL-10) and corticosteriods as well as the inhibition of Interleukins 1 and 6 (IL-1 and IL-6)²⁴. Expression of these three cytokines, TNF-α, IL-6 and IL-1, alters the metabolism of the swine resulting in less than optimal weight gain, development and health²⁵.

Not all mannans have immunostimulatory activity. The mannans including disaccharide through hexasaccharide, released by weak alkaline degradation of the cetyltrimethulammonium bromide (CTAB) extraction of Candida albicans, do not demonstrate any immunostimulatory activity. In fact, these small mannans are potent inhibitors of lymphoproliferation²⁶.

Although various research has investigated the supplementation of poultry diets, it has been previously unknown to supplement poultry diet with glucomannan compositions as a substitute for subtherapeutic doses of antibiotics.

### SUMMARY

The present instrumentalities overcome the problems outlined above and advance the art by providing a glucomannan composition that may be added to poultry diets for the benefit of poultry production. In one example, the glucomannan composition may be used to replace the subtherapeutic doses of antibiotics that are currently used in production poultry feeds.

Preferred forms of glucomannan include phosphorylated glucomannan polysaccharides containing a repetitive (20 to 160 times) structure of 9-13 monosaccharides linked with α1-6, α1-2 linkages, with mannose and glucose residues at a ratio of 8:1 to 12:1 mannose:glucose.

In other aspects, the phosphorylated glucomannan polysaccharaides may be administered to poultry in two basic forms, namely, phosphorylated glucomannan or phosphorylated glucomannan that is non-covalently linked to a protein. The phosphorylated glucomannan, with or without a non-covalently linked protein, may be adsorbed into a matrix. Without limitation, specific examples of absorption matrices include one or more inorganic salts, such as dihydrate calcium phosphate (CaHPO₄.2H₂O) and dihydrate calcium sulphate (CaSO₄.2H₂O). Phosphorylated glucomannan, with or without the non-covalently linked protein, absorbed or unabsorbed into a matrix, may be administered to the poultry, preferably, if the form of a dry powder thoroughly mixed into the nursery, grower or finishing feeds.

Benefits of administering the phosphorylated glucomannan compositions to animals, especially poultry, may include:
Increased animal weight gain;
Increased relative quantities of the beneficial bacteria in the animal;
Decreased relative quantities of malicious bacteria in the animal;
Increased uptake of beneficial minerals, nutrients and vitamins;
Increased uptake of zinc and copper;
Improved overall general health of the animal.
Replacement of subtherapeutic doses of antibiotics in animal feed; and/or
Reduced or eliminated subtherapeutic doses of antibiotics in animal feed.

A poultry diet may be supplemented by mixing a conventional poultry feed with a phosphorylated glucomannan polysaccharide in an effective amount to benefit poultry production, in order to provide a mixed poultry feed.

The phosphorylated glucomannan contains a repeating polysaccharide subunit that is repeated approximately n times of 1-6 and 1-2 linkages between and within mannose and glucose residues at a ratio of 12:1 mannose:glucose, were n ranges from 10 to 40. The value n may range from 10 to 20, from 20 to 30, from 30 to 40, or from 20 to 40, with n preferably being about 30.

The poultry feed may be provided as a liquid, gel or colloid, for example, in the nature of a vitamin or mineral supplement. In other forms of what is disclosed, the feed is prepared as solid food, preferably with a balance of nutrients that target poultry needs at a particular stage of poultry development.

### BRIEF DESCRIPTION OF THE DRAWINGS

FIG. 1 shows weight gain corresponding to respective intervals of an animal feeding study, where the results indicate that poultry production is facilitated by improved weight gain attributable to feeding of a phosphorylated glucomannan.

FIG. 2A shows the calibration curve for the Acute Phase Protein (APP) Analysis and the equation for calculation of the concentration of the unknown samples.

FIG. 2B displays a digital image of a typical result of a radial diffusion assay (FIG. 3B).

FIG. 3A through FIG. 3G show comparative body weight gains over time in a chicken feeding study at intervals of seven days (FIG. 3A), fifteen days (FIG. 3B), twenty-one days (FIG. 3C), twenty-eight days (FIG. 3D), thirty-five days (FIG. 3E), forty-two days (FIG. 3F), and forty-nine days (FIG. 3G), where the results indicate that poultry production is facilitated by improved weight gain attributable to feeding of test articles composed of phosphorylated glucomannan.

FIG. 4A through FIG. 4C provide a graphical summary of average daily feed intake in a chicken feeding study for the combined test articles (FIG. 4A), the test article CUP (FIG. 4B), and the test article CUPS (FIG. 4C), where the results indicate that poultry production is facilitated by improved weight gain attributable to feeding of two test articles composed of phosphorylated glucomannan.

FIG. 5A through FIG. 5C provide a graphical summary of current feed efficiency corrected for mortality in a chicken feeding study for the combined test articles (FIG. 5A), and the two test articles separated by treatment group (FIG. 5A and FIG. 5B), where the results indicate that poultry fed one of the basal feeds containing a concentration of one of the test articles have a corresponding improvement in the feed-to-gain ratio.

FIG. 6 shows a graphical representation of the average breast meat yield from the major and minor pectoral muscles from each of the treatment groups, indicating that the average yield of major and minor pectoral muscles correspond to a dose response curve for each of the test articles, and further indicating that doses of the test articles improve the average major and minor pectoral muscle yield.

FIG. 7A though FIG. 7R show the graphical representations of the different hematological and blood chemistry data for the chicken feeding study including uric acid (FIG. 7A); CPK, (FIG. 7B); globulin (FIG. 7C); chloride (FIG. 7D); potassium (FIG. 7E); sodium (FIG. 7F); phosphorous (FIG. 7G); calcium (FIG. 7H); cholesterol (FIG. 71); AST (FIG. 7J); albumin (FIG. 7K); protein (FIG. 7L); glucose (FIG. 7M); basophils (FIG. 7N); eosinophils (FIG. 70); percent combined lymphocytes-monocytes (FIG. 7P); percentage Hst/Poly's (FIG. 7Q); and WBC estimate per 103 mls of blood (FIG. 7R); this data indicating that poultry fed feed containing different doses of the test article have normal hematological and blood chemistry parameters as compared to the poultry fed the basal alone and with antibiotic.

FIG. 8 shows the graphical representation of the weekly average concentration of Acute Phase Proteins for the length of the study and based on the treatment group

### DETAILED DESCRIPTION

According to one embodiment, the phosphorylated glucomannan is provided as an additive to poultry feed that may be used at all stages of poultry development. The phosphorylated glucomannan may, for example, be added and mixed into the feed as a concentrated raw product, a concentrated raw product with a non-covalently attached protein, raw product absorbed into a matrix, and/or a concentrated raw product with a non-covalently attached protein absorbed into a matrix.

The phosphorylated glucomannan may be in the form of a dry powder that is capable of being added to or mixed with poultry feed. Dosing is by ratio or concentration that may vary according to the stage of poultry development to provide a benefit to the poultry by promoting the health of the poultry and replacing, reducing or eliminating the use of subtherapeutic doses of antibiotics in poultry nursery, grower, finisher and maintenance feeds.

Exemplary embodiments of various formulations include:
i) A dry powder comprised of the Phosphorylated Glucomannan Polysaccharides containing a repetitive (20 to 160 times) structure of 9-13 monosaccharides linked with α1-6, α1-2 linkages, with mannose and glucose residues at a ratio of 8:1 to 12:1 mannose:glucose mixed into poultry feed at a concentration, ratio, or dose that provides the general benefits of good health and weight gain to the poultry consuming the mixed feed.
ii) A dry powder comprised of Phosphorylated Glucomannan Polysaccharides containing a repetitive (20 to 160 times) structure of 9-13 monosaccharides linked with α1-6, α1-2 linkages, with mannose and glucose residues at a ratio of 8:1 to 12:1 mannose:glucose and a non-covalently linked protein mixed into poultry feed at a concentration, ratio, or dose that provides the general benefits of good health and weight gain to the poultry consuming the mixed feed.
iii) A dry powder comprised of the Phosphorylated Glucomannan Polysaccharides containing a repetitive (20 to 160 times) structure of 9-13 monosaccharides linked with α1-6, α1-2 linkages, with mannose and glucose residues at a ratio of 8:1 to 12:1 mannose:glucose and adsorbed into a matrix and mixed into poultry feed at a concentration, ratio, or dose that provides the general benefits of good health and weight gain to the poultry consuming the mixed feed.
iv) A dry powder comprised of Phosphorylated Glucomannan Polysaccharides containing a repetitive (20 to 160 times) structure of 9-13 monosaccharides linked with α1-6, α1-2 linkages, with mannose and glucose residues at a ratio of 8:1 to 12:1 mannose:glucose and a non-covalently linked protein and adsorbed into a matrix and mixed into poultry feed at a concentration, ratio, or dose that provides the general benefits of good health and weight gain to the poultry consuming the mixed feed.

A variety of poultry feeds are available commercially, and these may be formulated for various stages of poultry development. These feeds may be supplemented with minor amounts of a phosphorylated glucomannan, for example, as isolated from Candida utilis, to achieve the instrumentalities described herein. Other feed formulations may be provided by publicly available software, such as the User-Friendly Feed Formulation Program (“UFFDA”) based upon the book Animal Feed Formulation-Economics and Computer Applications, by G. M. Pesti and B. R. Miller, Chapman and Hall. The phosphorylated glucomannan mixed with this food to provide a dosage ranging from 1 to 5 mg of the phosphorylated glucomannan per kg of body weight in the poultry. The preferred dosage is 3 mg per kg of body weight Although higher doses may be used, such as doses of 20 mg/kg, the range from 1 mg to 5 mg per kg are generally minimal doses to achieve the desired effects.

### EXAMPLE 1

### Obtention of Candida Utilis Polysaccharide with Soy Protein Adsorbed on Calcium Phosphate

The following laboratory-scale example teaches by way of example how to purify a phosphorylated glucomannan polysaccharide. The polysaccharide contains a repetitive (20 to 160 times) structure of 9-13 monosaccharides linked with α1-6, α1-2 linkages, with mannose and glucose residues at a ratio of 8:1 to 12:1 mannose:glucose. The polysaccharide may be obtained, for example, using the process described in EP1163911 [this patent has not been awarded yet and it is under discussion], which is incorporated by reference, and describes the alternative use of soy or castor beans which are optionally omitted.

The method of isolating phosphorylated glucomannan polysaccharides commences, for example, by soaking soybeans in water to provide soaked soybeans. These are ground to provide ground material and combined with Candida utilis, water, and a first salt to provide an incubation mixture. The incubation mixture is incubated with stirring or agitation for extraction of the polysaccharide to provide a supernatant fluid. The supernatant is concentrated by filtration with a cutoff of about 20 kDa. A second salt is added together with a low molecular weight ketone to form a precipitate. The precipitate is dried to yield an isolated polysaccharide product.

In various aspects, the drying step is preferably performed at a temperature not more than 55° C. to avoid product degradation. The first salt is preferably a manganese salt, such as MnSO₄.H₂O. The incubation mixture may be provided with an amount of camphor that is miscible with the aqueous phase, and with heating to a temperature of from 30° C. to 40° C. Concentration may be staged, for example, using an initial stage of filtering to remove cellular debris, ultrafiltration to the 20 kDA cutoff to produce a concentrate of at least 1/10 the initial volume of the supernatant, and diafiltration of the concentrate against water in amount at least ten times the volume of the concentrate. The second salt is preferably a calcium salt, such as calcium chloride, where also the low molecular weight ketone is preferably acetone. The precipitate may be combined with an adsorption salt to stabilize the final product. Suitable adsorption salts include, for example, calcium phosphate (CaHPO₄.2H₂O) and/or dihydrate calcium sulphate (CaSO₄.2H₂O). The resulting isolated polysaccharide may be formulated by mixing with an animal feed carrier in a dosage formulation that is effective to reduce growth of non-beneficial microorganisms in the digestive tract of a predetermined animal.

In one embodiment, starting materials include commercial pasteurized and spray-dried standard food grade Candida utilis that is subjected to the preferred process described below:
1.1 Weigh approximately 100 g of soy bean seeds. Soak them for 24 hrs in water.
1.2 Wash the seeds several times with water.
1.3 Grind the seeds in a mortar or a mincer.
1.4 Prepare an aqueous solution of 21 containing 6.25 g/l of MnSO₄.H₂O at a temperature of 37° C. Add, stirring in a magnetic stirrer, 0.21 g/l of MnO₂, 0.6 g/l camphor, 62.5 g/l of desiccated C. utilis and 12.5 g/l of the seed milling.
1.5 Incubate in orbital stirrer at 37° C. and 200 rpm 2 to 5 hours, until the concentration of the polysaccharide is between 2 to 4 g/l.
1.6 Cool to a temperature less than 25° C., allow to stand, separate the supernatant and filter through a Hyflo®/Standar super cell® with a filter candle.
1.7 Concentrate the filtrated supernatant by ultrafiltration with a cut off of 20 kDa to a 1/10 of the original volume.
1.8 Diafiltrate the concentrate against at least 10 times of its volume of water.
1.9 Add, under stirring, calcium chloride to the concentrate/diafiltrate to a end concentration of 60 mM. Let, under stirring, 30 minutes.
1.10 Add, under stirring, calcium phosphate to a end concentration similar to three times the polysaccharide concentration. Let, under stirring, 15 minutes.
1.11 Add, under stirring, acetone to an end concentration of 40% (v/v).
1.12 Filter through nylon and separate the precipitate.
1.13 Dry the precipitate in a vacuum oven at temperature not higher than 55° C.

The above process is scalable to industrial level and implies an improvement respect to the prior art in the following points:
a) cobalt chloride is advantageously not needed.
b) filtration replaces centrifugation where filtration is a less expensive and more scalable process.
c) the former lyophilization is replaced either by precipitation or by adsorption on a salt, such as calcium phosphate, with precipitation. This renders a more stable product, due to the stabilizing action of the calcium phosphate.

### EXAMPLE 2

### Pilot Feeder Animal Study

Chicken feed studies were performed on a contract basis between a requesting agency and a testing agency. The study was commissioned using two different test articles, namely: (1) glucomannan and (2) glucomannan plus a non-covalently linked protein, for example soy bean proteins. Each test articles was mixed separately into chicken starter and grower feeds at X mg/kg body weight, where x is 1, 3 or 20. Mixing the test articles into the chicken feeds can occur as either a part of the chicken feed production process or mixed into the chicken feeds through mechanical means and processes post production of the chicken feed. A study of this type shows that chickens fed either test article performed better than chickens fed feed containing no antibiotic, and as well as or better than chickens fed feed containing antibiotic. Antibiotic (Bacitracin, BMD 60) was added to the basal diet at the concentration of one (1) lb/ton of diet (60 mgs of Bacitracin per 909 kg). Parameters used to compare chickens fed feed containing test articles at concentrations of X mg/kg body weight, where x is 1, 3 or 20 to chickens fed feed containing antibiotic or no antibiotic included are: total weight gain, weekly weight gain, the ratio of feed to gain, mortality, carcass weight, breast meat weight, bacterial flora, blood chemistry, peripheral blood cell populations and the response of acute phase proteins.

The basal grower feed used in this study included the materials shown in Table 2, mixed with the phosphorylated glucomannan as indicated below.

The feeder chicken feed used in this study included the materials shown Table 3, mixed with the phosphorylated glucomannan as indicated below.

### Experimental Design Study Summary

The two test articles, Candida utilis phosphoglucomannan and Candida utilis phosphoglucomannan-soy bean proteins, are mixed prior to study initiation with a carrier, such as (CaHPO₄.2H₂O) and/or dihydrate calcium sulphate (CaSO₄.2H₂O). The negative control is considered to have 0 mg test article and antibiotic/kg diet, it is the basal chicken feed. The test articles are titrated into the negative control feed at levels to approximate 1, 3, and 20 mg of active test article/kg body weight. BMD 60¹ is added to the negative control diet at one pound per ton diet, thus, there are 8 treatment groups.
¹ BMD 60 contains Bacitracin at 60 mg/lbs. BMD 60 produced by Carl S. Akey, Inc. PO Box 5002, Lewisburg, Ohio 45338.

Male Ross x Ross broiler chickens (250 count) were ordered from a hatchery and were received on Day 1 of life. Broilers were acclimated for 7 days to an environment of feed and water ad litium and a room temperature of 80° F. with the temperature under the provided heat lamp of approximately 95° F. During the acclimation period, light was provided for approximately 24 hours per day and the ventilation was by forced air designed to provide in excess of 10 air exchanges per hour. Pen bedding was an approximate mixture of 50/50 of fresh pine shavings and pine shavings that had been previously used for broiler chicken bedding. Criteria for broiler inclusion and exclusion was broiler chicks in good health with no outwardly obvious signs of illness or deformation were included in the study and any broiler showing any sign of illness or deformation was removed from the trail.

On Day 0 of the study, post 7 days of acclimation, all birds were individually weighed and wing banded. Wing band number and body weights were placed into a Microsoft® Excel 2002 SP-2. The “Rand” and “Rank” functions were used to generate and assign random numbers (Rand) for each broiler and to assign broilers into treatment groups (Rank) by random number. Each treatment group was divided into 2 pens of 13 birds, designated Replicate A and Replicate B. The eight rations are fed ad libitum to the assigned treatment groups of 2 pens of 13 birds each for the duration of the study. During days 0 to 7 of the study, the room temperature was gradually decreased to approximately 72° F. This temperature was maintained until termination of the study. Body weight, feed consumption and feed efficiency were measured weekly and feed efficiency was corrected for any mortality. Mortalities were recorded and a determination of the cause of death was provided, post necropsy, by a trained avian specialist and/or an Avian Veterinarian.

Blood was collected weekly from 3 predetermined birds from Replicate A of each treatment group and submitted for CBC/Chemistries. Additionally, six (6) birds per treatment group (3 per pen) were sacrificed on Day 3 for CBC & Chemistries. At the conclusion of the study, the birds were sacrificed and gross pathology was performed by a licensed Veterinarian Avian specialist, gut samples were taken from three of the birds from each treatment group (Replicate B) and sent off to a Veterinary diagnostic lab to determine levels of Salmonella spp. and Campylobacter spp. present. In addition, major and minor pectorals were removed from all birds in each treatment group and weighed. Major and minor pectoral weights' were used to calculate average weight of major and minor pectoral muscles for each treatment group.

Justification for Route, Duration

It is common practice in the poultry industry, as well as the swine and beef industries, to include antibiotics and other growth promoters in the feed of the bird; this is the most cost efficient method. Alternative, but less desirable routes of administration include through the drinking water, and injection or inoculation. The duration of the study is designed to mimic a standard growth phase commonly found in the broiler chicken industry, which is between 6 to 8 weeks.

Justification for Test Animal Selection

These products are designed to replace antibiotics at sub therapeutic levels in broiler chickens. Economically, chickens are a major source of food protein throughout the world. In the United States, broiler chickens (whole and parts) are consumed at the rate of 9 billion per year and in Europe, at the rate of 5 billion per year. Further, these animals are quick growers and are raised in conditions that are very amenable to controlled environments.

Justification for Number of Animals

The number of animals utilized in the first study is considered to be the minimum necessary to evaluate the effects of the test articles in comparison to sub therapeutic doses of antibiotics in broiler chickens. From the results of the first study, the statistical parameters were used to perform a “power-of-the-test” to calculate the number of birds needed in a second study to ensure that the number of animals utilized would be sufficient to support the desired significance (alpha=0.05) of the study.

Justification for Dose Selection

The current dose levels for the two test articles are 0, ˜1 mg active test article/kg body weight, ˜3 mg active test article/kg body weight, and ˜20 mg active test article/kg body weight. These doses are considered to be safe doses for the two test articles (Candida utilis phosphoglucomannan and Candida utilis phosphoglucomannan-soy bean proteins).

### STUDY OUTLINE

Effective Area

Sub Therapeutic Antibiotic Replacement

Test Articles

Test Article 1 (Candida utilis phosphoglucomannan adsorbed in calcium phosphate). Candida utilis phosphoglucomannan 10-13% (w/w), dihydrated calcium phosphate 87-90% (w/w). This compound may be prepared by Industrial Farmaceutica Cantabria and provided to a test agency prior to study initiation.

Test Article 2 (Candida utilis phosphoglucomannan-soy bean proteins adsorbed in calcium phosphate-calcium sulphate). Candida utilis phosphoglucomannan-Soy bean proteins 5-10% (w/w), dihydrated calcium phosphate dihydrated calcium sulphate 90-95% (w/w). This compound is prepared by Industrial Farmaceutica Cantabria and provided to the test location prior to study initiation.

Unless otherwise noted, the identity, strength, purity, composition, stability and method of synthesis, fabrication and/or derivation of each batch of the test and control articles is documented by the test agency before its use in the study. This documentation is maintained by the test agency.

Archival Samples

An archival sample from each lot of test article is taken and stored in the Archives of the test agency, pending shipment to Industrial Farmaceutica Cantabria.

Preparation of Test Diets

Given the desired dose (approximate) levels of the test articles of 1, 3, and 20 mg active test article/kg body weight, the average ratio of grams feed intake/day/Kg body weight is taken from the NRC (1994) for the bird of age 1 to 3 weeks and 3 to 9 weeks. This value is used to determine the mg total product/Kg feed to mix for each treatment group and time period. The following Table 1 outlines the values that may be used for each treatment:

TABLE 1
DOSING STUDY

| | | | | Dose Levels | | | |
|---|---|---|---|---|---|---|---|
| | | Pos Cont. | | (Active Article per Test Article) | | | |
| | | addition | | | ˜3 mg | | |
| Test | | mg/kg | Neg | ˜1 mg per | per Kg | ˜20 mg per | |
| Article | | feed | Cont. | Kg Feed | Feed | Kg Feed | Comments |
| Candida | N = 26* | 499.4 | 0.0 | 88.63 | 265.9 | 1772.64 | 0-3 Week |
| utilis-Phos | | | | | | | |
| (10% | | | | | | | |
| active) | | | | | | | |
| Candida | N = 26* | — | — | 125.24 | 375.71 | 2504.72 | 3-7 Week |
| utilis-Phos | | | | | | | |
| (10% | | | | | | | |
| active) | | | | | | | |
| Candida | N = 26* | — | — | 177.26 | 531.79 | 3545.28 | 0-3 Week |
| utililis- | | | | | | | |
| Phos + Soy | | | | | | | |
| (5% | | | | | | | |
| active) | | | | | | | |
| Candida | N = 26* | — | — | 250.47 | 751.42 | 5009.44 | 3-7 Week |
| utililis- | | | | | | | |
| Phos + Soy | | | | | | | |
| (5% | | | | | | | |
| active) | | | | | | | |

*Six birds per Tx group are sacrificed on day 3, N = 20 per Tx group thereafter

Table 1 shows the dosing levels for each test article. Table 1 lists the amount of test article to add based on the required dose level of the active article consumed per kilogram of feed consumed. This information is used, along with calculated feed intake from the NRC (1994) to determine the actual amount (mg) of the test articles to add to each diet, as shown in Table 4:

Commercial feeds were purchased for use in mixing with the test articles, as shown in Tables 2 and 3.

Tables 1 and 2 show the content of test article feeds that were used in the study.

TABLE 2
COMMERCIAL FEED
CHICK & FRYER
CRUMBLE
ACTIVE DRUG INGREDIENT

| | |
|---|---|
| Amprolium | 0.0125% |

GUARANTEED ANALYSIS

| | |
|---|---|
| Crude Protein, not less than | 20.0% |
| Lysine, note less than | 1.0% |
| Methlonine, not less than | 0.3% |
| Crude Fat, not less than | 3.5% |
| Crude Fiber, not less than | 5.5% |
| Calcium (Ca), not less than | 0.8% |
| Calcium (Ca), not more than | 1.3% |
| Phosphorus (P), not less than | 0.6% |
| Salt (NaCl), not less than | 0.3% |
| Salt (NaCl), not more than | 0.8% |

INGREDIENTS
Grain Products, Plant Protein Products, Processed Grain By-
Products, Animal Protein Products, Salt, DL-Methionine, Calcium
Carbonate, Dicalcium Phosphate, Manganous Oxide, Zinc Oxide,
Ferrous Sulfate, Copper Sulfate, Ethylenediamine Dihydriodide,
Sodium Selenite, Manganese Proteinate, Vitamin A Acetate D-
Activated Animal Sterol (source of Vitamin D-3), Vitamin E
Supplement, Vitamin B-12 Supplement, Riboflavin Supplement,
Niacin Supplement, D-Calcium Pantothonate, Menadione Sodium
Bisulfate Complex, Folic Acid, Biotin, Thiamine Mononitrate,
Pyridoxine Hydrochloride, Lignin Sulfonate (a pellet binder)

TABLE 3
COMMERCIAL FEED
CHICK GROWER A
ACTIVE DRUG INGREDIENT

| | |
|---|---|
| Amprolium | 0.0125% |

GUARANTEED ANALYSIS

| | |
|---|---|
| Crude Protein, not less than | 15.0% |
| Lysine, note less than | 0.65% |
| Methlonine, not less than | 0.25% |
| Crude Fat, not less than | 3.0% |
| Crude Fiber, not less than | 6.0% |
| Calcium (Ca), not less than | 0.6% |
| Calcium (Ca), not more than | 1.1% |
| Phosphorus (P), not less than | 0.55% |
| Salt (NaCl), not less than | 0.2% |
| Salt (NaCl), not more than | 0.7% |

INGREDIENTS
Grain Products, Plant Protein Products, Processed Grain By-
Products, Forage Products, Protein Products, Dicalcium Phosphate,
Calcium Carbonate, Lignin Sulfonate (a pellet binder), Salt,
Manganous Oxide, Zinc Oxide, Ferrous Sulfate, Copper Sulfate,
Ethylenediamine Dihydriodide, Sodium Selenite, Manganese
Proteinate, Vitamin A Acetate D-Activated Animal Sterol (source
of Vitamin D-3), Vitamin E Supplement, Vitamin B-12
Supplement, Riboflavin Supplement, Niacin Supplement, D-
Calcium Pantothonate, Menadione Sodium Bisulfate Complex,
Folic Acid, Biotin, Thiamine Mononitrate, Pyridoxine
Hydrochloride, DL-Methionine.

TABLE 4
AMOUNT OF TEST MATERIAL IN FEED.

| | | Bird Age 1-3 Week | Bird Age 3-7 Week |
|---|---|---|---|
| | Feed Consumed NRC | (Study Days 0-14) | (Study Days 15-49) |
| | (1994) | Test Article Addition | Test Article Addition |
| Treatment (N = 26*) | Bird Ages (0-3 wk/3-7 wk) | (mg per feed) | (mg per feed) |
| Candida utilis-Phos | 18.24 Kg/81.72 Kg | 1616.61 | 10,234.61 |
| 1 mg/Kg | | | |
| Candida utilis-Phos | 18.24 Kg/81.72 Kg | 4850.02 | 30,703.02 |
| 3 mg/Kg | | | |
| Candida utilis-Phos | 18.24 Kg/81.72 Kg | 32,332.95 | 204,685.71 |
| 20 mg/Kg | | | |
| Candida utilis- | 18.24 Kg/81.72 Kg | 3233.22 | 20,468.41 |
| Phos + Soy | | | |
| 1 mg/Kg | | | |
| Candida utilis- | 18.24 Kg/81.72 Kg | 9699.84 | 61,406.04 |
| Phos + Soy | | | |
| 3 mg/Kg | | | |
| Candida utilis- | 18.24 Kg/81.72 Kg | 64,665.91 | 409,371.43 |
| Phos + Soy | | | |
| 20 mg/Kg | | | |

*Six birds per Tx group are sacrificed on day 3, N = 20 per Tx group thereafter

Analysis of Test Diets

Due to the nature of the test articles there is currently no accurate methodology to quantitate the amount of test article or its activity in the test diets other than an empirical study, for example, as described herein.

Preparation of Facilities

Prior to the receipt of the poultry the facility is cleaned and sanitized removing all organic matter. Each pen is set up so as to isolate it from all other pens; this is done in order to prevent possible cross contamination among pens. Each pen is provided one Plason gravity flow watering device, one brooding lamp and one 25 lbs. gravity flow feeder. The pen floors are covered in a heavy gauge plastic and provided with a mixture (50/50) of new wood shavings and shavings previously used by broiler chickens.

Acquisition of Animals

250 Ross x Ross male broiler chickens are obtained on commercial order from Hoover Hatchery, P.O. Box 200,205 Chickasaw St. Rudd, Iowa 50471.

General Husbandry

Poultry are housed in an environmentally controlled room at the test agency for the duration of the study.

The poultry are provided with the lighting conditions shown in Table 5.

TABLE 5
STUDY LIGHTING CONDITIONS

| Growth Phase | Photo Period |
|---|---|
| 0 to 7 days | 24 hours lights on |
| (Study Days −6 to 0) | |
| 8-55 days | 16 hours lights on/8 hours lights |
| (Study Days 1 to 48) | off |

Prior to the arrival of the birds the facility is adjusted to approximately 27° C. (80.7° F.). All brooding lamps are lowed such that the temperature directly under each lamp is ˜35° C. (95° F.). This is done in order to allow the broiler chickens a temperature gradient in each pen as birds are poikilotherms until they develop adult feathering.

At day 0 of the study the heat lamps are removed. At this time 2 daily observations are made in order to insure the birds are maintained at proper temperature.

During the acclimation phase (days −6 to −1) all birds are fed ad libitum the negative control diet, containing no test article or antibiotic.

Each pen is initially fed 20 lbs. of the designated ration. The feed intake is observed daily and feed is weighed and added as necessary in order to insure the birds are maintained on ad libitum feeding. (Need section on feed titrations)

TABLE 6
SUMMARY OF TREATMENT GROUPS

| Test Article | Concentration in Diet | | |
|---|---|---|---|
| Candida utilis-Phos | 1 mg active/kg | 3 mg | 20 mg |
| (10% active) | (CUP 1) | active/kg | active/kg |
| | | (CUP 3) | (CUP 20) |
| Candida utililis-Phos + Soy | 1 mg active/kg | 3 mg | 20 mg |
| (5% active) | (CUPS 1) | active/kg | active/kg |
| | | (CUPS 3) | (CUPS 20) |
| BMD 60 | 60 mg active/kg | | |
| | (BMD 60) | | |
| Additive Free | 0 mg active/kg | — | — |
| | (W/O) | | |

Animal Identification

At approximately day −6 of the experimental phase all birds are wing banded with a unique number identification in the right wing.

Animal Selection at Day 0 of Experimental Phase

At day 0 of the experimental phase all birds are individually weighed. Poultry selection and randomization procedures is conducted by test agency personnel (other than the Investigator or Co-Investigator) using Microsoft® (D Excel 2002 (10.4524.4219) SP-2. Random numbers are generated using the “Rand” function of Excel and are captured using the “copy/paste special/values” commands. The “Rank” function in Excel is used to assign poultry to groups within blocks by random number. In addition, single factor ANOVA data analysis (a=0.05) in Excel is used to assess the outcomes of randomizations for homogeneity of variance (F statistic<F critical value) between groups. ANOVA is conducted for body weight between pens.

Bird Selection for Blood Draw

Each pen has an additional 3 birds (N total birds =13 per pen) included at Day 0 to provide 3 birds per pen (6 per treatment) on Day 3 for sacrifice and blood collection. Selection of the 3 birds for the Day 3 sacrifice is by a random number assignment. All thirteen birds in each pen receive a random number generated in Excel. The three birds with the highest random numbers within each pen are selected for the Day 3 collection.

Ten (10) birds remain in each pen to complete the study. Three (3) birds from the Replicate A pen of each treatment group are selected for blood draw each week of the experimental phase. Within Replicate A, birds are selected for blood collection based on their random numbers. The birds with the lowest 3 random numbers are drawn on weeks 1, 4, and 7, the next 3 lowest on weeks 2, and 5, and the next 3 lowest on weeks 3 and 6. The tenth bird within the replicate is considered an extra bird.

Unused Test Articles

Unused test article mixtures and containers are returned to the requesting agency. Collection equipment used in the study are autoclave and disposed of in the biohazard/sharps solid waste stream at the test agency.

### Test Animals

Species

Broiler Chickens

Supplier

Hoover Hatchery,

P.O. Box 200,

205 Chickasaw St. Rudd, Iowa 50471.

Animal Requirements/Specifications

| Total* | Males* | Females* |
|---|---|---|
| 208 | 208 | 0 |

0-55 days of age

Acclimation Period

At this stage, the broiler chickens begin acclimation to study conditions at about 5 to 7 days prior to the initiation of the trial. During acclimation, all poultry are checked for viability twice daily. Prior to assignment to study, all poultry are examined to ascertain suitability for study by a staff veterinarian.

At approximately day −7 of the study clinical observations are made by a staff veterinarian for each bird. Any bird that is found abnormal is rejected from the study.

At approximately day −6 of the study all birds are individually wing banded with a unique numerical identification in the right wing. At approximately day 28 the birds are given an additional wing tag in the left wing, this is the same number as was placed in the right wing at day approximately −6.

### Animal Care and Husbandry

Facilities Management/Animal Husbandry

Currently acceptable practices of good animal husbandry are followed, e.g., as shown in the Guide for the Care and Use of Laboratory Animals; National Academy Press, 1996. The test agency, for example, Sinclair Research Center, Inc. (SRC), may be fully accredited to perform contract studies by the Association for Assessment and Accreditation of Laboratory Animal Care International (AAALAC). The Guide for the Care and Use of Laboratory Animals; recommends 2 square feet for birds up to 3 kg. These birds may average, for example, 2.5 kg at the termination of the study. The contemplated test facilities offer approximately 22 square feet per pen, this provides adequate spacing for 10 birds per pen

Veterinary Care

Poultry are monitored by the technical staff for any conditions requiring possible veterinary care. If any such conditions are identified, a staff veterinarian is notified for an examination and evaluation.

Environmental Conditions

During days −6 to 0 the birds are given approximately 24 hours of light. From day 0 to study termination the birds are given approximately 16 hours of light and 8 hours of dark.

Temperature is monitored in accordance with standard procedure at the test agency. From days −6 to day 0, each pen is provided with a heating lamp. The temperature under this lamp is maintained at approximately 95° F. (35° C.). The room is set at approximately 80° F. (˜27° C.), this temperature is monitored and recorded daily. From day 7 to 14 the room temperature is decreased gradually to approximately 72° F. This temperature is maintained for the duration of the study.

Humidity is monitored in accordance with standard procedure at the test agency, but is not controlled.

Housing

The broiler chickens are housed in groups of 10 in individual floor pens in an environmentally controlled room for the duration of the study.

Feed

Birds are allowed ad libitum feeding. From days −6 to 0 all birds are given the negative control diet (containing no test articles or antibiotics). From day 0 forward each pen is given its respective diet ad libitum.

Water

Clean, fresh water from an on-site deep well is available ad libitum during the study. Water is provided via Plason gravity flow waterers.

Bedding

An approximate mixture of 50/50 (v/v) of fresh pine shavings and pine shavings that have been previously used for broiler chicken bedding is used in this trial. The purpose of the litter contamination is to increase the pathogen burden in the test birds to better reflect the normal farm husbandry condition. It is also desirable to have a pressure of infection to determine the efficacy of the test article.

Feed Analysis

Nutritional certification of batches of feed provided by the manufacturer (via manufacturer's bag label) is included in the raw data. There are no known contaminants in the food which are expected to interfere with the objectives of this study.

Water Analysis

A copy the test agency's most recent water analysis is included with the raw data. There are no known contaminants which are expected to interfere with the objectives of this study.

### In-Life Evaluations Observations

Body Weight Gain

Each bird is weighed once a weekly, this information is recorded in the study records.

Feed Intake

Feed is weighed out prior to feeding. All feed added to a pen is weighed and recorded in the study records. Once weekly the feeders are weighed and weights recorded in order to determine feed disappearance.

Environmental

Once daily the minimum, maximum and current temperature and humidity are recorded in the study records.

Mortality

Mortality is recorded daily for each pen in the study book. The body weight is recorded for each mortality and recorded in the study book.

Euthanasia

The broiler chickens are euthanized by an intravenous overdose of sodium pentobarbital (390 mg/mL)/sodium phenytion (50 mg/mL) at 0.22 mL/Kg, followed by cervical dislocation (e.g., as SRC SOP PR. 04.01).

Blood Collection.

Blood is collected for determination of CBC with differential and Chemistries on Study Days 3, 7, and weekly thereafter. The samples are collected by test agency personnel and sent to a suitable analytical company, such as Antech Diagnostics for analysis. For the day 3 draw the birds are sacrificed and blood is collected via a direct heart draw. From Days 7 on the blood is collected from the brachial artery. For the CBC approximately 1 mL of whole blood is drawn using a drop for the blood smear and the rest drawn into an EDTA microtainer for storage and reuse. The differential for the CBC is automated. The analytical chemistry requires approximately 0.50 mL serum from each bird.

Bacteriology

On day 48 of the study 3 birds from each pen are sacrificed for gut collection. A sample of the small intestine is collected from each bird, from the ileum-cecal junction to the Meckel's Diverticulum. Sub samples from this portion of the small intestine is taken and sent to Antech Diagnostics Laboratories to determine Salmonella spp. and Campylobacter spp. counts. This data is recorded in the study records.

Breast Meat Yield

On day 48 of the study all birds are sacrificed and the right pectorals major and minor are removed and weighed. This data is recorded in the study records.

Gross Necropsy/Gross Pathology

On day 48 of the study all birds are sacrificed and a staff Veterinarian performs a gross necropsy and gross pathology. These results are included in the study report.

TABLE 7
SUMMARY OF DATA COLLECTED DURING THE STUDY

| | | | Day of |
|---|---|---|---|
| Event | Study Day | Date | Week |
| Study Initiation-(Randomization into pens) | 0 | 20-Jun-05 | Mon |
| Blood collection (3 Birds/Pen) | 3 | 23-Jun-05 | Thu |
| Blood collection/Body Weight/Feed Intake (All pens) | 7 | 27-Jun-05 | Mon |
| Blood collection/Body Weight/Feed Intake (All pens) | 15 | 5-Jul-05 | Tue |
| Switch to Grower Feed | 15 | 5-Jul-05 | Tue |
| Blood collection/Body Weight/Feed Intake (All pens) | 21 | 11-Jul-05 | Mon |
| Blood collection/Body Weight/Feed Intake (All pens) | 28 | 18-Jul-05 | Mon |
| Blood collection/Body Weight/Feed Intake (All pens) | 35 | 25-Jul-05 | Mon |
| Blood collection/Body Weight/Feed Intake (All pens) | 42 | 1-Aug-05 | Mon |
| Blood collection/Body Weight/Feed Intake (All pens) | 49 | 8-Aug-05 | Mon |
| Study Termination (Necropsy (all pens)/Gut Collection | 49 | 8-Aug-05 | Mon |
| (3 Birds/Treatment Group)) | | | |

Archiving of Records and Specimens

All data documenting experimental details and study procedures and observations are recorded and maintained as raw data. At the completion of the study, all reports and study specific original raw data, and copies of certain study related facility data are reported. An exact copy of the report and raw data is maintained in the test agency's archives for a period of at least 1 year after submission of the signed final report. All plasma samples are shipped to the test requester. The test requestor is responsible for retaining samples of the test article.

Statistical Analysis

ANOVA statistical analysis is performed on study data including Body Weight Gain, Feed Consumption, Feed Efficiency corrected for mortality, and breast meat yield. Alpha is set at 0.05.

A study according to the above protocol has been completed. Data from the study supports the preferred embodiment and the claims, as shown in Tables 4-16.

### Result

Definition of Effects to be Achieved and Clinical Endpoints:

For this study results for the Total Body Weight Gain on a per treatment group basis, Weekly Feed Intake, Current Feed Efficiency Corrected for Mortality, Percent Breast Meat Yield, Weekly Blood Chemistry and Hematology, Acute Phase Protein analysis and Bacteriology results were reported.

Schedule of Events

TABLE 8
SCHEDULE OF EVENTS

| | Study | Approximate | Day of |
|---|---|---|---|
| Event | Day | Date | Week |
| 7-Day Acclimation Period Initiates | −7 | 13-Jun-05 | Mon |
| Received/Acclimation begins | −7 | 13-Jun-05 | Mon |
| | −6 | 14-Jun-05 | Tue |
| | −5 | 15-Jun-05 | Wed |
| | −4 | 16-Jun-05 | Thu |
| | −3 | 17-Jun-05 | Fri |
| | −2 | 18-Jun-05 | Sat |
| | −1 | 19-Jun-05 | Sun |
| Study Initiation-(Randomization into pens) | 0 | 20-Jun-05 | Mon |
| | 0 | 20-Jun-05 | Mon |
| | 1 | 21-Jun-05 | Tue |
| | 2 | 22-Jun-05 | Wed |
| Blood collection (3 Birds/Pen) | 3 | 23-Jun-05 | Thu |
| | 4 | 24-Jun-05 | Fri |
| | 5 | 25-Jun-05 | Sat |
| | 6 | 26-Jun-05 | Sun |
| Blood collection (3 Birds/Trmt)/Body | 7 | 27-Jun-05 | Mon |
| Weight/Feed Intake) | 8 | 28-Jun-05 | Tue |
| | 9 | 29-Jun-05 | Wed |
| | 10 | 30-Jun-05 | Thu |
| | 11 | 1-Jul-05 | Fri |
| | 12 | 2-Jul-05 | Sat |
| | 13 | 3-Jul-05 | Sun |
| | 14 | 4-Jul-05 | Mon |
| Blood collection/Body | 15 | 5-Jul-05 | Tue |
| Weight/Feed Intake (Grower) | 16 | 6-Jul-05 | Wed |
| | 17 | 7-Jul-05 | Thu |
| | 18 | 8-Jul-05 | Fri |
| | 19 | 9-Jul-05 | Sat |
| | 20 | 10-Jul-05 | Sun |
| Blood collection (3 Birds/Trmt)/Body | 21 | 11-Jul-05 | Mon |
| Weight/Feed Intake | 22 | 12-Jul-05 | Tue |
| | 23 | 13-Jul-05 | Wed |
| | 24 | 14-Jul-05 | Thu |
| | 25 | 15-Jul-05 | Fri |
| | 26 | 16-Jul-05 | Sat |
| | 27 | 17-Jul-05 | Sun |
| Blood collection (3 Birds/Trmt)/Body | 28 | 18-Jul-05 | Mon |
| Weight/Feed Intake | 29 | 19-Jul-05 | Tue |
| | 30 | 20-Jul-05 | Wed |
| | 31 | 21-Jul-05 | Thu |
| | 32 | 22-Jul-05 | Fri |
| | 33 | 23-Jul-05 | Sat |
| | 34 | 24-Jul-05 | Sun |
| Blood collection (3 Birds/Trmt)/Body | 35 | 25-Jul-05 | Mon |
| Weight/Feed Intake | 36 | 26-Jul-05 | Tue |
| | 37 | 27-Jul-05 | Wed |
| | 38 | 28-Jul-05 | Thu |
| | 39 | 29-Jul-05 | Fri |
| | 40 | 30-Jul-05 | Sat |
| | 41 | 31-Jul-05 | Sun |
| Blood collection (3 Birds/Trmt)/Body | 42 | 1-Aug-05 | Mon |
| Weight/Feed Intake | 43 | 2-Aug-05 | Tue |
| | 44 | 3-Aug-05 | Wed |
| | 45 | 4-Aug-05 | Thu |
| | 46 | 5-Aug-05 | Fri |
| | 47 | 6-Aug-05 | Sat |
| | 48 | 7-Aug-05 | Sun |
| Blood collection (3 Birds/Trmt)/Body | 49 | 8-Aug-05 | Mon |
| Weight/Feed Intake | | | |
| Study Termination(Necropsy, | 49 | 8-Aug-05 | Mon |
| all birds/Gut Collection, 3 Birds/Trmt) | | | |

Animal Randomization and Selection

TABLE 9
RANDOMIZATION DATA WITH ANOVA SUMMARY

| Bird | Body Wt. | | | |
|---|---|---|---|---|
| ID | (g) | Block | Rand | Pen |
| 31 | 97.34 | 1 | 0.053777 | 1 |
| 223 | 100.02 | 2 | 0.02944 | 1 |
| 222 | 106.35 | 3 | 0.025437 | 1 |
| 143 | 108.77 | 4 | 0.019595 | 1 |
| 189 | 110.37 | 5 | 0.074094 | 1 |
| 135 | 113.87 | 6 | 0.117349 | 1 |
| 146 | 114.5 | 7 | 0.007343 | 1 |
| 9 | 117.65 | 8 | 0.037771 | 1 |
| 127 | 119.63 | 9 | 0.046796 | 1 |
| 195 | 122.48 | 10 | 0.024885 | 1 |
| 207 | 122.84 | 11 | 0.071303 | 1 |
| 75 | 127.99 | 12 | 0.043872 | 1 |
| 97 | 133.82 | 13 | 0.038292 | 1 |
| 165 | 99.92 | 1 | 0.126233 | 2 |
| 220 | 102.61 | 2 | 0.060536 | 2 |
| 33 | 106.41 | 3 | 0.029687 | 2 |
| 6 | 108.31 | 4 | 0.120615 | 2 |
| 129 | 109.67 | 5 | 0.273451 | 2 |
| 241 | 111.48 | 6 | 0.14819 | 2 |
| 45 | 116.26 | 7 | 0.010355 | 2 |
| 72 | 116.82 | 8 | 0.047779 | 2 |
| 118 | 118.89 | 9 | 0.083727 | 2 |
| 19 | 121.59 | 10 | 0.057057 | 2 |
| 156 | 123.72 | 11 | 0.27006 | 2 |
| 138 | 128.02 | 12 | 0.08199 | 2 |
| 201 | 133.07 | 13 | 0.063147 | 2 |
| 213 | 96.6 | 1 | 0.143023 | 3 |
| 32 | 103.15 | 2 | 0.21064 | 3 |
| 130 | 104.65 | 3 | 0.143773 | 3 |
| 5 | 106.95 | 4 | 0.147854 | 3 |
| 11 | 109.64 | 5 | 0.305576 | 3 |
| 169 | 113.13 | 6 | 0.261661 | 3 |
| 39 | 113.88 | 7 | 0.045045 | 3 |
| 60 | 117.85 | 8 | 0.120614 | 3 |
| 79 | 119.81 | 9 | 0.107469 | 3 |
| 114 | 121.22 | 10 | 0.058445 | 3 |
| 206 | 125.45 | 11 | 0.273296 | 3 |
| 47 | 128.62 | 12 | 0.187555 | 3 |
| 247 | 132.92 | 13 | 0.068715 | 3 |
| 23 | 94.14 | 1 | 0.257532 | 4 |
| 235 | 101.09 | 2 | 0.308088 | 4 |
| 46 | 104.82 | 3 | 0.207019 | 4 |
| 78 | 109.03 | 4 | 0.169195 | 4 |
| 204 | 111.08 | 5 | 0.317118 | 4 |
| 116 | 112.2 | 6 | 0.400082 | 4 |
| 238 | 115.77 | 7 | 0.149616 | 4 |
| 205 | 117.5 | 8 | 0.148542 | 4 |
| 197 | 120.32 | 9 | 0.26247 | 4 |
| 109 | 121.41 | 10 | 0.07456 | 4 |
| 243 | 124.12 | 11 | 0.341746 | 4 |
| 128 | 128.81 | 12 | 0.193057 | 4 |
| 167 | 132.42 | 13 | 0.269466 | 4 |
| 110 | 97.16 | 1 | 0.305452 | 5 |
| 95 | 101.01 | 2 | 0.336432 | 5 |
| 85 | 105.88 | 3 | 0.283548 | 5 |
| 37 | 107.27 | 4 | 0.288122 | 5 |
| 1 | 111.05 | 5 | 0.344375 | 5 |
| 29 | 112.74 | 6 | 0.407462 | 5 |
| 227 | 116.24 | 7 | 0.323165 | 5 |
| 25 | 117.8 | 8 | 0.158438 | 5 |
| 158 | 119.72 | 9 | 0.283362 | 5 |
| 148 | 121.52 | 10 | 0.132456 | 5 |
| 218 | 125.38 | 11 | 0.425625 | 5 |
| 82 | 129.02 | 12 | 0.2285 | 5 |
| 160 | 133.96 | 13 | 0.279741 | 5 |
| 64 | 98.54 | 1 | 0.355488 | 6 |
| 163 | 100.88 | 2 | 0.344005 | 6 |
| 89 | 104.96 | 3 | 0.293583 | 6 |
| 177 | 108.25 | 4 | 0.403078 | 6 |
| 248 | 110.32 | 5 | 0.368923 | 6 |
| 208 | 112.22 | 6 | 0.411835 | 6 |
| 115 | 114.69 | 7 | 0.323772 | 6 |
| 179 | 116.52 | 8 | 0.178832 | 6 |
| 91 | 120.16 | 9 | 0.303075 | 6 |
| 57 | 121.18 | 10 | 0.164632 | 6 |
| 239 | 123.48 | 11 | 0.431734 | 6 |
| 230 | 129.17 | 12 | 0.31165 | 6 |
| 187 | 131.65 | 13 | 0.347289 | 6 |
| 80 | 95.12 | 1 | 0.43228 | 7 |
| 249 | 102.48 | 2 | 0.511236 | 7 |
| 22 | 104.89 | 3 | 0.343917 | 7 |
| 151 | 106.5 | 4 | 0.435151 | 7 |
| 212 | 110.35 | 5 | 0.399843 | 7 |
| 88 | 111.79 | 6 | 0.452621 | 7 |
| 250 | 115.66 | 7 | 0.425494 | 7 |
| 162 | 117.84 | 8 | 0.22787 | 7 |
| 233 | 120.47 | 9 | 0.329235 | 7 |
| 200 | 121.33 | 10 | 0.202137 | 7 |
| 132 | 124.71 | 11 | 0.438491 | 7 |
| 111 | 129.48 | 12 | 0.339409 | 7 |
| 7 | 133.09 | 13 | 0.374561 | 7 |
| 117 | 99.11 | 1 | 0.456847 | 8 |
| 42 | 100.94 | 2 | 0.683243 | 8 |
| 63 | 106.44 | 3 | 0.414004 | 8 |
| 61 | 106.6 | 4 | 0.503405 | 8 |
| 73 | 109.33 | 5 | 0.419878 | 8 |
| 12 | 112.56 | 6 | 0.470644 | 8 |
| 155 | 115.08 | 7 | 0.447237 | 8 |
| 40 | 117.92 | 8 | 0.229471 | 8 |
| 94 | 118.99 | 9 | 0.490928 | 8 |
| 219 | 122.61 | 10 | 0.254279 | 8 |
| 35 | 123.86 | 11 | 0.468753 | 8 |
| 228 | 127.49 | 12 | 0.350355 | 8 |
| 181 | 130.95 | 13 | 0.382765 | 8 |
| 71 | 94.1 | 1 | 0.614097 | 9 |
| 26 | 102.66 | 2 | 0.735946 | 9 |
| 183 | 104.98 | 3 | 0.486848 | 9 |
| 70 | 108.71 | 4 | 0.649303 | 9 |
| 86 | 111.12 | 5 | 0.626479 | 9 |
| 16 | 111.46 | 6 | 0.536599 | 9 |
| 216 | 115.04 | 7 | 0.524512 | 9 |
| 76 | 117.45 | 8 | 0.239582 | 9 |
| 173 | 118.76 | 9 | 0.583023 | 9 |
| 180 | 121.02 | 10 | 0.369204 | 9 |
| 157 | 123.12 | 11 | 0.477911 | 9 |
| 4 | 127.35 | 12 | 0.366372 | 9 |
| 188 | 130.85 | 13 | 0.416472 | 9 |
| 144 | 95.58 | 1 | 0.630455 | 10 |
| 113 | 103.64 | 2 | 0.762659 | 10 |
| 210 | 105.44 | 3 | 0.547362 | 10 |
| 106 | 108.79 | 4 | 0.654715 | 10 |
| 137 | 109.88 | 5 | 0.775497 | 10 |
| 150 | 111.53 | 6 | 0.583595 | 10 |
| 196 | 115.36 | 7 | 0.526648 | 10 |
| 214 | 118.07 | 8 | 0.428918 | 10 |
| 237 | 118.74 | 9 | 0.58786 | 10 |
| 28 | 121.35 | 10 | 0.3974 | 10 |
| 186 | 124.25 | 11 | 0.495235 | 10 |
| 202 | 129.21 | 12 | 0.382561 | 10 |
| 131 | 134.86 | 13 | 0.547111 | 10 |
| 90 | 96.9 | 1 | 0.797484 | 11 |
| 193 | 104.63 | 2 | 0.7664 | 11 |
| 101 | 105.85 | 3 | 0.672139 | 11 |
| 153 | 109.09 | 4 | 0.748928 | 11 |
| 87 | 109.48 | 5 | 0.82911 | 11 |
| 126 | 111.41 | 6 | 0.695482 | 11 |
| 198 | 116.18 | 7 | 0.587935 | 11 |
| 190 | 117 | 8 | 0.450161 | 11 |
| 199 | 119.03 | 9 | 0.615196 | 11 |
| 182 | 121.45 | 10 | 0.466645 | 11 |
| 18 | 125.87 | 11 | 0.545551 | 11 |
| 141 | 130.13 | 12 | 0.385428 | 11 |
| 209 | 132.68 | 13 | 0.635758 | 11 |
| 112 | 96.85 | 1 | 0.843558 | 12 |
| 168 | 101.68 | 2 | 0.826155 | 12 |
| 221 | 106.18 | 3 | 0.801631 | 12 |
| 107 | 108.31 | 4 | 0.771555 | 12 |
| 145 | 111.22 | 5 | 0.858318 | 12 |
| 54 | 111.7 | 6 | 0.696899 | 12 |
| 136 | 114.3 | 7 | 0.596049 | 12 |
| 211 | 116.52 | 8 | 0.517446 | 12 |
| 232 | 118.46 | 9 | 0.627978 | 12 |
| 159 | 120.49 | 10 | 0.48106 | 12 |
| 154 | 123.39 | 11 | 0.550883 | 12 |
| 120 | 128.05 | 12 | 0.436879 | 12 |
| 191 | 133.63 | 13 | 0.772307 | 12 |
| 43 | 94.01 | 1 | 0.878567 | 13 |
| 8 | 102.46 | 2 | 0.877232 | 13 |
| 124 | 104.89 | 3 | 0.821819 | 13 |
| 229 | 106.54 | 4 | 0.826728 | 13 |
| 225 | 111.37 | 5 | 0.87702 | 13 |
| 103 | 112.41 | 6 | 0.839045 | 13 |
| 74 | 116.34 | 7 | 0.87413 | 13 |
| 164 | 118.22 | 8 | 0.579775 | 13 |
| 121 | 120.05 | 9 | 0.666337 | 13 |
| 15 | 121.83 | 10 | 0.566318 | 13 |
| 194 | 125.12 | 11 | 0.631322 | 13 |
| 50 | 126.74 | 12 | 0.503784 | 13 |
| 174 | 131.85 | 13 | 0.788928 | 13 |
| 62 | 96.55 | 1 | 0.9054 | 14 |
| 51 | 103.3 | 2 | 0.907703 | 14 |
| 172 | 105.66 | 3 | 0.852913 | 14 |
| 123 | 108.27 | 4 | 0.841758 | 14 |
| 119 | 110.91 | 5 | 0.891776 | 14 |
| 93 | 112.8 | 6 | 0.893906 | 14 |
| 133 | 116.32 | 7 | 0.909368 | 14 |
| 83 | 118.14 | 8 | 0.611309 | 14 |
| 20 | 120.27 | 9 | 0.710422 | 14 |
| 134 | 122.58 | 10 | 0.60993 | 14 |
| 27 | 123.22 | 11 | 0.774294 | 14 |
| 152 | 128.17 | 12 | 0.803989 | 14 |
| 105 | 131.76 | 13 | 0.938354 | 14 |
| 65 | 99.74 | 1 | 0.909455 | 15 |
| 21 | 102.81 | 2 | 0.939721 | 15 |
| 142 | 106.17 | 3 | 0.878382 | 15 |
| 66 | 108.08 | 4 | 0.861439 | 15 |
| 215 | 109.53 | 5 | 0.902733 | 15 |
| 175 | 113.76 | 6 | 0.899772 | 15 |
| 38 | 114.86 | 7 | 0.980991 | 15 |
| 53 | 117.9 | 8 | 0.762246 | 15 |
| 55 | 119.65 | 9 | 0.791984 | 15 |
| 108 | 120.55 | 10 | 0.700514 | 15 |
| 52 | 123.98 | 11 | 0.834653 | 15 |
| 102 | 127.15 | 12 | 0.875079 | 15 |
| 166 | 135.74 | 13 | 0.944018 | 15 |
| 69 | 99.76 | 1 | 0.966492 | 16 |
| 44 | 101.43 | 2 | 0.9407 | 16 |
| 139 | 106.01 | 3 | 0.932967 | 16 |
| 36 | 109.05 | 4 | 0.873251 | 16 |
| 92 | 109.63 | 5 | 0.947367 | 16 |
| 49 | 113.6 | 6 | 0.979502 | 16 |
| 13 | 115.75 | 7 | 0.98472 | 16 |
| 147 | 117.96 | 8 | 0.84403 | 16 |
| 149 | 120.35 | 9 | 0.928151 | 16 |
| 236 | 120.85 | 10 | 0.960407 | 16 |
| 41 | 124.33 | 11 | 0.917445 | 16 |
| 81 | 129.2 | 12 | 0.927316 | 16 |
| 30 | 132.92 | 13 | 0.987869 | 16 |

ANOVA: SUMMARY of Body Weight Randomization by Treatment
Group

| Groups | Count | Sum | Average | Variance |
|---|---|---|---|---|
| 1 | 2 | 229.6585 | 114.8292 | 0.096124 |
| 2 | 2 | 229.4015 | 114.7008 | 0.0648 |
| 3 | 2 | 229.8692 | 114.9346 | 0.272663 |
| 4 | 2 | 230.1585 | 115.0792 | 0.005472 |
| 5 | 2 | 230.25 | 115.125 | 0.139636 |
| 6 | 2 | 229.6169 | 114.8085 | 0.198935 |
| 7 | 2 | 230.2692 | 115.1346 | 0.007764 |
| 8 | 2 | 230.0562 | 115.0281 | 0.354741 |

ANOVA of Body Weight Randomization by Treatment Group

| Source of | | | | | | |
|---|---|---|---|---|---|---|
| Variation | SS | df | MS | F | P-value | F crit |
| Between | 0.368551 | 7 | 0.05265 | 0.369431 | 0.896436 | 3.50046 |
| Groups | | | | | | |
| Within | 1.140136 | 8 | 0.142517 | | | |
| Groups | | | | | | |
| Total | 1.508686 | 15 | | | | |

Indicates similar body weight among treatment groups at the initiation of the study.

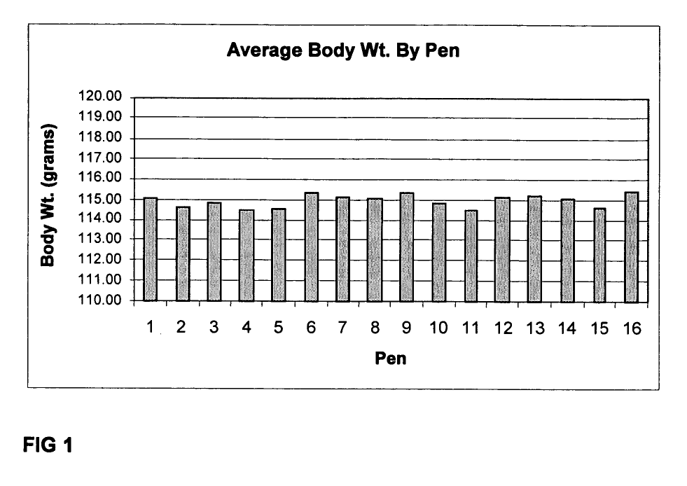

FIG. 1 presents a graphical representation of the data tabulated in Table 9. Graphical and statistically, individuals have been randomly selected and assigned to pens and treatments with very little or no bias.

TABLE 10
WEEKLY BLOOD DRAW SELECTION
Weekly Bird Selection for Blood Collection

| | 27-Jun | 5-Jul | 11-Jul | 18-Jul | 25-Jul | 1-Aug | 8-Aug |
|---|---|---|---|---|---|---|---|
| Pen # | Bird ID | Bird ID | Bird ID | Bird ID | Bird ID | Bird ID | Bird ID |
| 9 | 157 | 188 | 16 | 157 | 188 | 180 | 157 |
| 9 | 216 | 70 | 71 | 216 | 70 | 16 | 216 |
| 9 | 183 | 173 | 4 | 183 | 173 | 71 | 183 |
| 10 | 113 | 131 | 202 | 113 | 131 | 186 | 113 |
| 10 | 106 | 237 | 210 | 106 | 237 | 202 | 106 |
| 10 | 214 | 28 | 137 | 214 | 28 | 210 | 214 |
| 11 | 87 | 199 | 193 | 87 | 199 | 182 | 190 |
| 11 | 190 | 126 | 141 | 190 | 126 | 193 | 101 |
| 11 | 101 | 209 | 153 | 101 | 209 | 141 | 199 |
| 12 | 145 | 120 | 136 | 145 | 107 | 136 | 145 |
| 12 | 112 | 54 | 211 | 112 | 54 | 211 | 112 |
| 12 | 191 | 154 | 221 | 191 | 154 | 221 | 191 |
| 13 | 164 | 43 | 50 | 74 | 43 | 15 | 74 |
| 13 | 8 | 103 | 15 | 8 | 103 | 8 | 43 |
| 13 | 124 | 74 | 8 | 124 | 50 | 124 | 103 |
| 14 | 133 | 134 | 62 | 133 | 134 | 62 | 133 |
| 14 | 172 | 20 | 123 | 172 | 20 | 123 | 172 |
| 14 | 105 | 119 | 93 | 105 | 119 | 133 | 105 |
| 15 | 52 | 55 | 142 | 52 | 55 | 142 | 52 |
| 15 | 38 | 66 | 108 | 38 | 66 | 108 | 38 |
| 15 | 102 | 175 | 65 | 102 | 175 | 65 | 102 |
| 16 | 81 | 149 | 139 | 149 | 236 | 13 | 41 |
| 16 | 69 | 41 | 92 | 41 | 92 | 147 | 236 |
| 16 | 147 | 236 | 13 | 147 | 139 | 149 | 139 |

Mortalities

TABLE 11
MORTALITIES
Mortalities

| | | Body | Date of | | |
|---|---|---|---|---|---|
| Bird ID | Pen | Wt. (g) | Mort | Findings/Cause of Death | Examiner |
| | | | | Emaciation and Dehydradtion, | |
| 72 | 2 | 519 | Jul. 19, 2005 | Osteomalacia | Dr. Bermudez |
| 206 | 3 | 253 | 1-Jul-05 | Colibacillosis | Dr. Bermudez |
| 11 | 3 | 438 | 24-Jul-05 | Splay Leg | Dr. Bermudez |
| 197 | 4 | 673 | Jul. 15, 2005 | Ascites | Dr. Bermudez |
| 128 | 4 | 2270 | Aug. 4, 2005 | Colibacillosis | Dr. Bermudez |
| 91 | 6 | 2387 | Jul. 30, 2005 | Colibacillosis | Dr. Bermudez |
| 212 | 7 | 1298 | Jul. 20, 2005 | Colibacillosis | Dr. Bermudez |
| 219 | 8 | 255 | 3-Jul-05 | Colibacillosis | Dr. Bermudez |
| 155 | 8 | 417 | Jul. 16, 2005 | Splay Leg | Dr. Bermudez |
| 87 | 11 | 2387 | Jul. 30, 2005 | pulmonary edema | Dr. Bermudez |
| 120 | 12 | 1490 | 21-Jul-05 | cardiopulmonary failure | Dr. Bermudez |
| 194 | 13 | 139 | 23-Jun-05 | Blocked Intestine | Eric Blair |
| 121 | 13 | 390 | 29-Jun-05 | Sudden Death Syndrome | Dr. Bermudez |
| 164 | 13 | 242 | 4-Jul-05 | Colibacillosis | Dr. Bermudez |
| | | | | undetermined musuloskeletal | |
| 93 | 14 | 1100 | Jul. 30, 2005 | problem/dehydration and inanition | Dr. Bermudez |
| 69 | 16 | 246 | 27-Jun-05 | Rapid Blood Draw | Dr. Bermudez |
| 81 | 16 | 331 | 4-Jul-05 | Colibacillosis | Dr. Bermudez |
| 44 | 16 | 1885 | 24-Jul-05 | cardiopulmonary failure | Dr. Bermudez |

Total Body Weight Gain:

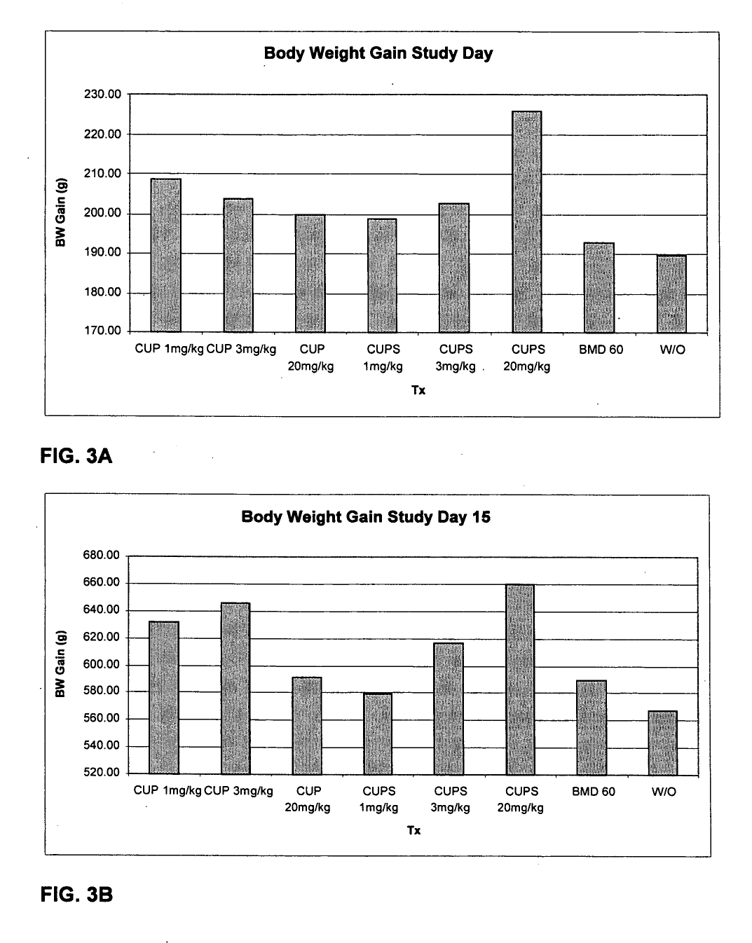

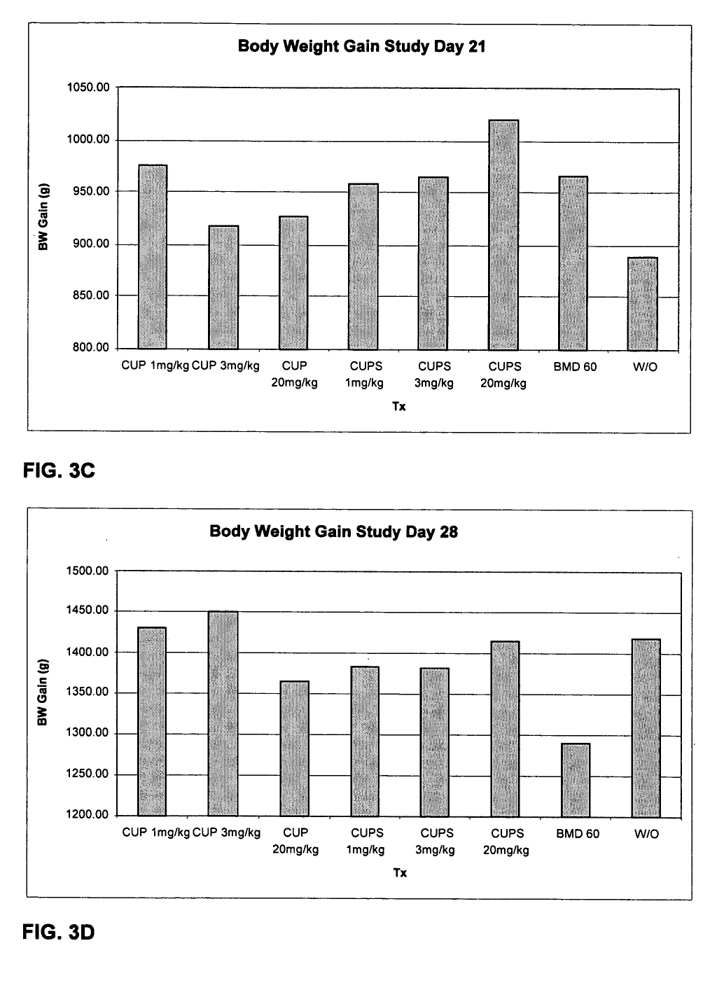

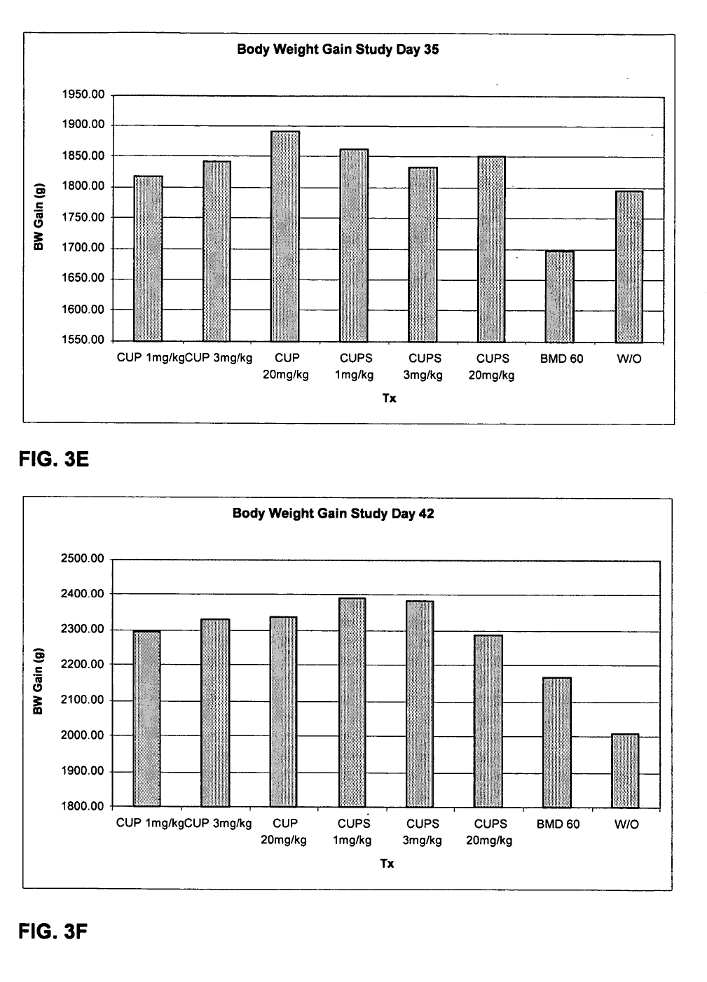

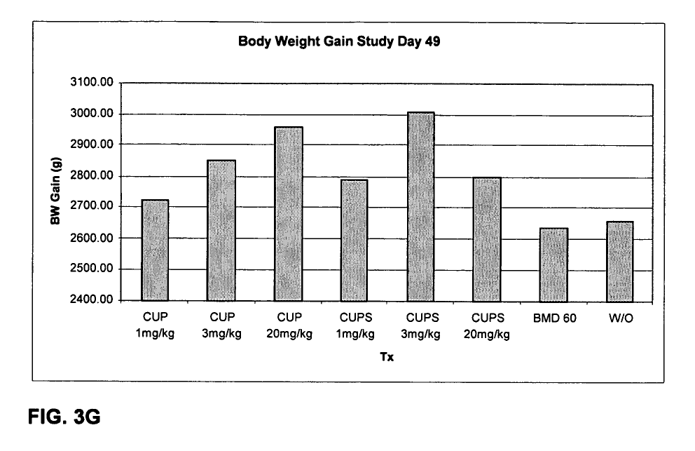

At measurement period 0-15 days, there was a statistically significant difference for mean body weight gain between the W/O and CUPS 20 (W/O: 567.6 g, CUPS 20: 659.0 g). At measurement period 0-42, there were statistically significant differences for mean body weight gain between the W/O and CUP 3, CUP 20, CUPS 1 and CUPS 3 (W/O: 1997.0 g, CUP 3: 2330.6 g, CUP 20: 2335.8 g, CUPS 1: 2395.6 g, CUPS 3: 2383.5 g). At measurement period 0-49, there were statistically significant differences for mean body weight gain between the BMD 60 and CUP 20 and CUPS 3 (BMD 60: 2510.4 g, CUP 20: 2966.6 g, CUPS 3: 3005.7 g). There were no other statistically significant differences in mean body weight gain between compounds and dose groups throughout the remaining measurement periods (Table 12). Graphical displays of total body weight for measurement period 0-7 days is presented in FIG. 3A, measurement period 0-15 days is presented in FIG. 3B, measurement period 0-21 is presented in FIG. 3C, measurement period 0-28 days is presented in FIG. 3D, measurement period 0-35 days is presented in FIG. 3E, measurement period 0-42 days is presented in FIG. 3F and measurement period 0-49 days as presented in FIG. 3G.

TABLE 12
SUMMARY OF TOTAL BODY WEIGHT GAIN PER
TREATMENT GROUP¹

| | (Days of Study) | | | | | | |
|---|---|---|---|---|---|---|---|
| Treatments | 7 | 15 | 21 | 28 | 35 | 42 | 49 |
| CUP 1 mg/kg | 208.64 | 631.64^{ab} | 975.64 | 1430.09 | 1817.11 | 2293.92^{ab} | 2720.47^{ab} |
| CUP 3 mg/kg | 203.82 | 645.73^{ab} | 917.97 | 1450.82 | 1840.55 | 2328.86^{a} | 2849.94^{ab} |
| CUP 20 mg/kg | 199.89 | 590.44^{ab} | 927.54 | 1365.28 | 1891.17 | 2335.79^{a} | 2959.20^{a} |
| CUPS 1 mg/kg | 198.76 | 579.29^{ab} | 958.16 | 1383.09 | 1862.90 | 2390.14^{a} | 2788.44^{ab} |
| CUPS 3 mg/kg | 202.81 | 616.71^{ab} | 964.96 | 1382.61 | 1832.01 | 2383.45^{a} | 3005.67^{a} |
| CUPS 20 mg/kg | 225.92 | 658.97^{a} | 1021.02 | 1416.02 | 1852.50 | 2288.26^{ab} | 2796.44^{ab} |
| BMD 60 | 192.82 | 589.28^{ab} | 966.00 | 1289.95 | 1696.45 | 2166.06^{ab} | 2634.28^{b} |
| W/O | 189.70 | 567.60^{b} | 889.05 | 1418.25 | 1795.42 | 2010.07^{b} | 2653.88^{ab} |

¹Values with different letters are statistically (p < 0.05) different.

Weekly Feed Intake

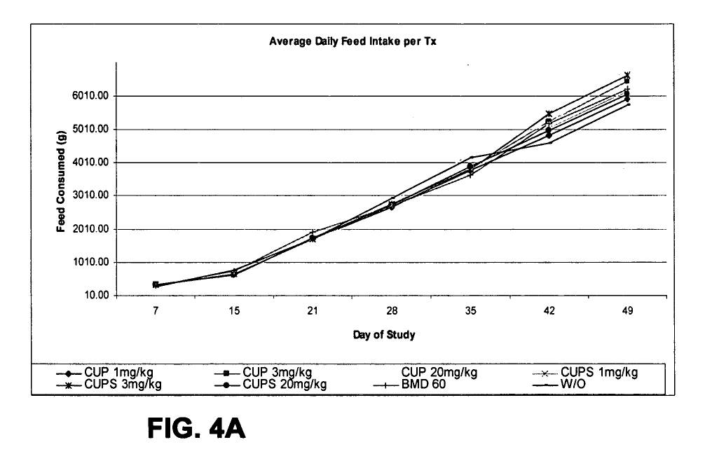

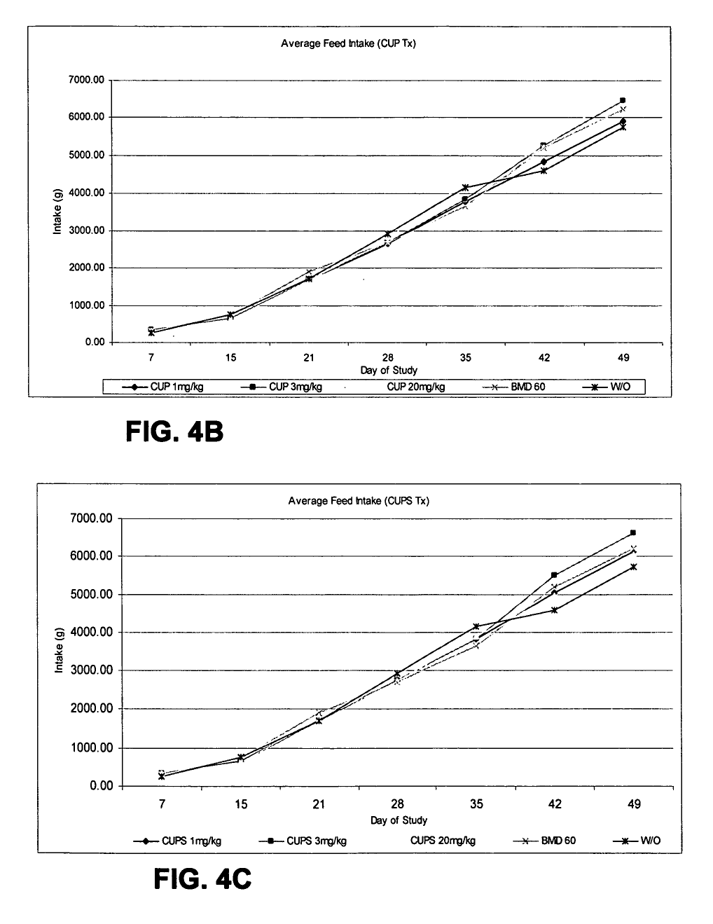

At Days 0-7, there was a statistically significant difference for average feed intake between W/O and CUP 1 mg/kg, CUP 3 mg/kg, CUP 20 mg/kg, CUPS 1 mg/kg, CUPS 3 mg/kg and CUPS 20 mg/kg (Table 13). There were no other statistically significant differences in average feed intake between compounds and dose groups throughout the remaining measurement periods. Average daily feed intake is graphical displayed for all treatment groups (FIG. 4A) and for the two test articles, CUP and CUPS, FIGS. 4B and 4C, respectively.

TABLE 13
AVERAGE WEEKLY FEED INTAKE PER BIRD FOR EACH
TREATMENT GROUP¹

| | Day of Study | | | | | | |
|---|---|---|---|---|---|---|---|
| Groups | 7 | 15 | 21 | 28 | 35 | 42 | 49 |
| CUP 1 mg/kg | 324.65^{a} | 640.45 | 1697.85 | 2665.60 | 3795.29 | 4827.66 | 5904.98 |
| CUP 3 mg/kg | 328.45^{a} | 647.41 | 1707.41 | 2692.72 | 3848.66 | 5246.59 | 6448.31 |
| CUP 20 mg/kg | 324.50^{a} | 633.70 | 1665.25 | 2799.98 | 4272.04 | 5190.28 | 6794.87 |
| CUPS 1 mg/kg | 321.93^{a} | 652.91 | 1739.64 | 2751.44 | 3811.29 | 5050.49 | 6144.64 |
| CUPS 3 mg/kg | 331.90^{a} | 643.70 | 1715.65 | 2754.25 | 3806.45 | 5487.94 | 6618.78 |
| CUPS 20 mg/kg | 338.55^{a} | 635.00 | 1748.75 | 2719.55 | 3897.33 | 4968.13 | 6054.27 |
| BMD 60 | 312.55^{ab} | 739.66 | 1919.13 | 2690.15 | 3648.25 | 5191.16 | 6222.70 |
| W/O | 265.90^{b} | 762.80 | 1717.65 | 2938.19 | 4152.93 | 4592.15 | 5735.40 |

¹Values with different letters are statistically (p < 0.05) different.

Current Feed Efficiency Corrected for Mortality

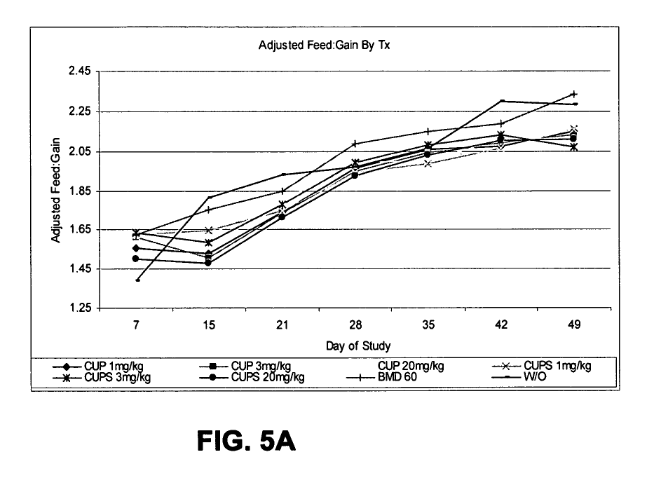

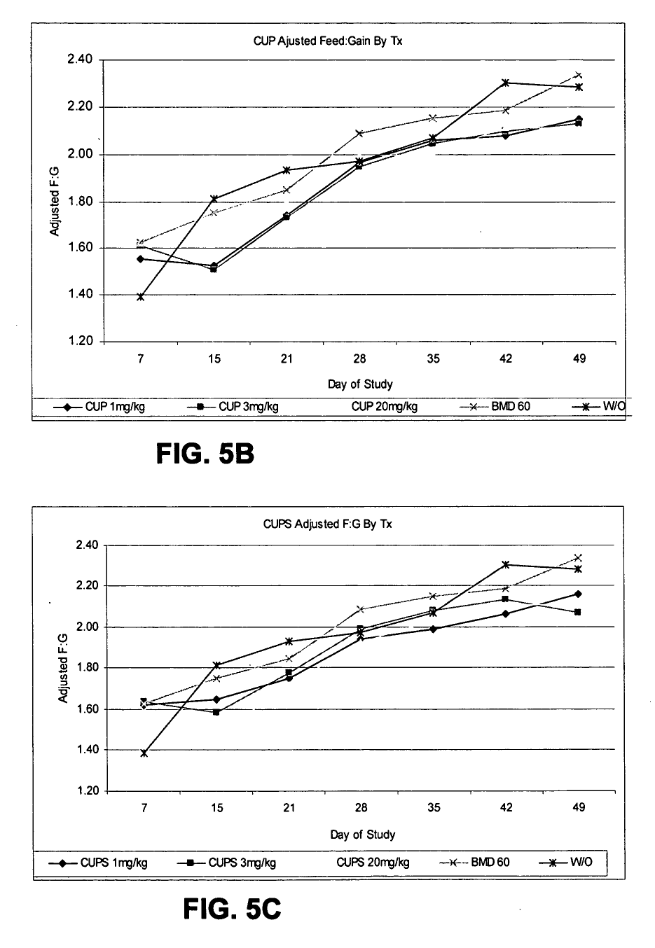

At measurement periods 0-15 and 0-21, there were statistically significant differences for average feed efficiency between the W/O and the CUPS 20 mg/kg preparation (Table 14). There were no other statistically significant differences in average feed efficiency between compounds and dose groups throughout the remaining measurement periods. This data has been graphically summarized in FIG. 5, where FIG. 5A displays the combined data from all treatment groups and FIGS. 5B & 5C display the data from the test articles.

TABLE 14
CURRENT FEED EFFICIENCY CORRECTED FOR
MORTALITY¹

| | Day of Study | | | | | | |
|---|---|---|---|---|---|---|---|
| Groups | 7 | 15 | 21 | 28 | 35 | 42 | 49 |
| CUP 1 mg/kg | 1.56 | 1.53^{ab} | 1.74^{ab} | 1.96 | 2.06 | 2.08 | 2.15 |
| CUP 3 mg/kg | 1.61 | 1.51^{ab} | 1.73^{ab} | 1.95 | 2.04 | 2.09 | 2.13 |
| CUP 20 mg/kg | 1.62 | 1.62^{ab} | 1.80^{ab} | 2.00 | 2.00 | 2.12 | 2.11 |
| CUPS 1 mg/kg | 1.62 | 1.65^{ab} | 1.75^{ab} | 1.94 | 1.99 | 2.07 | 2.16 |
| CUPS 3 mg/kg | 1.64 | 1.58^{ab} | 1.78^{ab} | 1.99 | 2.08 | 2.13 | 2.07 |
| CUPS 20 mg/kg | 1.50 | 1.48^{a} | 1.72^{a} | 1.92 | 2.03 | 2.11 | 2.11 |
| BMD 60 | 1.63 | 1.75^{ab} | 1.85^{ab} | 2.09 | 2.15 | 2.19 | 2.33 |
| W/O | 1.39 | 1.81^{b} | 1.93^{b} | 1.97 | 2.07 | 2.30 | 2.28 |

¹Values with different letters are statistically (p < 0.05) different.

Percent Breast Meat Yield

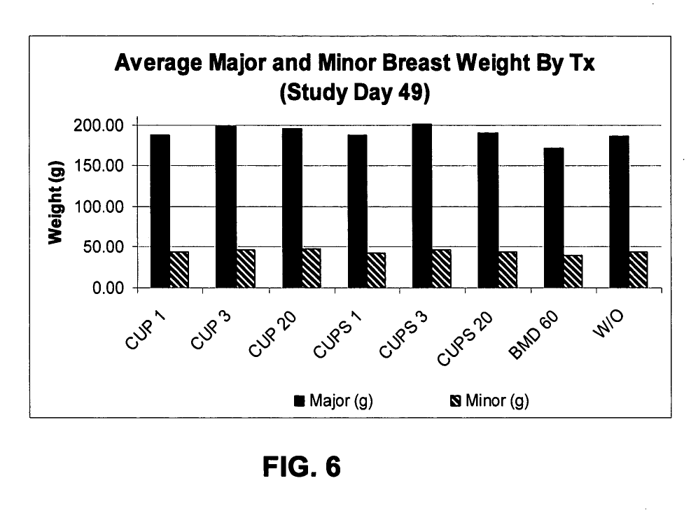

For the breast meat yield data there were no significant (p<0.05) differences found among any of the treatment groups. Table 15 summarizes the Percent Breast Meat Yield for each treatment group for both the Major and Minor pectorals. This data has been graphically summarized in FIG. 6.

TABLE 15
PERCENT BREAST MEAT YIELD PER TREATMENT GROUP

| | Pectoralis | Pectoralis |
|---|---|---|
| Treatment | Major | Minor |
| CUP 1 | 6.58% | 1.55% |
| CUP 3 | 6.62% | 1.59% |
| CUP 20 | 6.27% | 1.52% |
| CUPS 1 | 6.39% | 1.46% |
| CUPS 3 | 6.40% | 1.50% |
| CUPS 20 | 6.52% | 1.53% |
| BMD 60 | 6.21% | 1.46% |
| W/O | 6.70% | 1.57% |

Blood Chemistry Statistical Results

At Day 7, there was a statistically significant difference for calcium between the CUP 3 and CUPS 20; phosphorus between W/O and BMD 60; serum potassium between the BMD 60 and CUP 3, CUP 20, CUPS 1, CUPS 3 and CUPS 20. At Day 15, there were statistically significant differences for serum uric acid concentration between CUPS 1 and BMD 60, CUP 3, CUPS 20 and CUPS 3. At Day 28, there were statistically significant differences for sodium concentration between CUP 20 and CUPS 1. At Day 42, there were statistically significant differences for serum uric acid concentration between the BMD 60 and W/O and BMD 60 and CUPS 3. On Day 49, there was a significant difference in glucose concentrations between CUPS 1 and CUP 3 and CUPS 1 and W/O. There were no other statistically significant differences in serum chemistry indices between the W/O or BMD 60 and other treatment compounds and dose groups throughout the remaining measurement periods (Table 16). While ANOVA identified statistically significant differences between treatment groups for Day 3 calcium concentration p=0.04), Day 7 sodium concentration p=0.03), Day 28 cholesterol (p=0.047), Day 35 albumin (p=0.03) and uric acid (p=0.03) concentrations and Day 42 AST concentration (p=0.03), there was insufficient statistical power to pair-wise identify the existence of any significant difference between specific individual treatment groups.

Blood Chemistry Clinical Results

Uric acid (UA) normal range is from 2.5 to 8.1 mg/dL in an adult chicken. In the current study there were a few significant variations in UA between treatment groups but all values reported are compatible with normal renal function. UA levels were a little higher than is normally found in adult chickens during the first week of the trial (23 Jun. 2005) and this may be due to the immature renal function in these young broilers and/or slight dehydration in all treatment groups at the time of sampling.

Overall, creatinine kinase (CK or CPK) levels were normal indicating normal skeletal muscle integrity throughout the study. CK levels were elevated in three treatment groups at the termination of the study (8 Aug. 2005) but these numerical differences were not statistically significant. Larger and heavier broiler chickens are likely to sit more and this muscle inactivity will result in necrosis of isolated muscle fibers. CK is a very sensitive indicator of muscle necrosis so a few birds in the pen that are large and less mobile will have a marked CK increase and this will skew the pen mean. Likewise, isolated broilers with right sided heart failure (ascites syndrome in broilers) might also have elevated CK levels due to myocardial necrosis.

Blood globulin levels range from 1.5 to 4.1 g/dL in adult chickens and 1.33 g/dL in reported in 3-week-old broilers (Ledoux et al, 1999). There were no statistically different differences in globulin levels in the current study and globulin levels are interpreted to be normal. Until the last time point in the study globulin levels were lower than the normal adult chicken range indicated above. Younger chickens will naturally have lower immunoglobulins in the serum because they have not been exposed to as many antigens.

Ledoux et al., (1999) report a blood chloride level of 110 mEq/L in normal 3-week-old broilers. This is similar to the mean chloride levels reported in the current study. There were no statistical differences between any treatments and all chloride levels were interpreted to be normal.

Blood potassium levels vary from 3.0 to 7.3 mEq/L in adult chickens. Blood potassium is slightly above this range (8.1 mEq/L) in the BMD 60 treatment group on Study Days 3 and 7. While this is an observation worth noting it does not appear to have any significance in the health of this treatment group. Otherwise, all blood potassium levels were within normal physiologic ranges and statistically significant differences are not physiologically significant.

Blood sodium levels vary from 131 to 171 mEq/L in adult chickens and mean blood sodium was 139 mEq/L in 3-week-broilers (Ledoux et al. 1999). Blood sodium levels in all treatment groups at all time points were within normal physiologic ranges and the statistical significant differences noted are not physiologically significant differences.

Blood phosphorus levels vary from 6.2 to 7.9 mg/dL in adult chickens and a mean value of 8.17 mg/dL is reported in normal 3-week-old broilers. At day 7 of the current study the W/O group had a statistically lower blood phosphorus level (5.73 mg/dL) than the BMD group (8.53 mg/dL). All blood phosphorus levels are interpreted to be within normal physiologic levels.

Blood calcium levels in adult chickens do not provide a normal range for broilers because hens in egg production have high blood calcium levels. Ledoux et al. (1999) report blood calcium levels of 9.36 mg/dL in 3-week-old broilers. Small statistically significant differences in blood calcium levels are reported at day 7 of this study. All blood calcium levels in all treatment groups throughout the study are interpreted to be within normal physiologic ranges.

Blood cholesterol levels vary from 86 to 211 mg/dL in adult chickens and mean blood cholesterol was 102 mg/dL in 3-week-broilers (Ledoux et al. 1999). Blood cholesterol levels in all treatment groups at all time points were within normal physiologic ranges and no statistical significant differences were noted between treatment.

Aspartate aminotransferase (AST) levels were in a normal range compared to other avian species (parrot and macaw) as reported in Avian Medicine: Principles and applications by Ritchie, Harrison and Harrison (1994). Blood AST levels in all treatment groups at all time points were within normal ranges and no statistical significant differences were noted between treatments. These results indicate normal hepatic integrity in all treatment groups.

Blood albumin levels vary from 1.3-2.8 g/dl in adult chickens and mean blood albumin was 1.26 g/dL in 3-week-broilers (Ledoux et al. 1999). Blood albumin levels were slightly below these normal ranges in all treatment groups during the beginning of the current study and then move into this normal range at the end of the study. This is interpreted to be a normal age related increase in blood albumin. This conclusion is supported by the fact that no statistically significant differences between any treatments were noted.

Blood protein levels vary from 3.3 to 5.5 g/dl in adult chickens and mean blood protein was 2.58 mg/dL in 3-week-broilers (Ledoux et al. 1999). Blood protein levels were slightly below these normal ranges in all treatment groups during the beginning of the current study and then move into this normal range at the end of the study. This is interpreted to be a normal age related increase in blood protein. This conclusion is supported by the fact that no statistically significant differences between any treatments were noted. Blood protein is simply the addition of albumin and globulin and similar trends are reported above with these constituents of total protein.

Blood glucose levels vary from 227 mg/dL to 300 mg/dL in adult chickens and mean blood glucose was 357 mg/dL in 3-week-broilers (Ledoux et al. 1999). The blood glucose levels in the current study were predominantly with in this range and the statistical differences noted at day 49 of the study are completely within normal physiologic ranges. Blood glucose levels are interpreted to be within normal physiologic limits within all treatment groups at all dates evaluated.

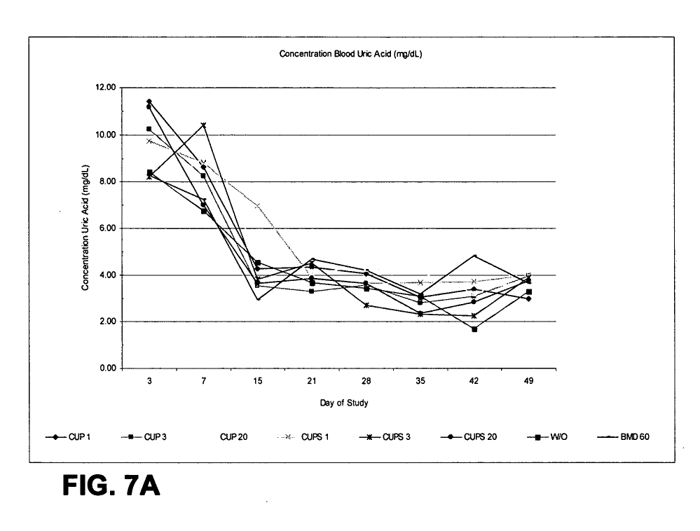

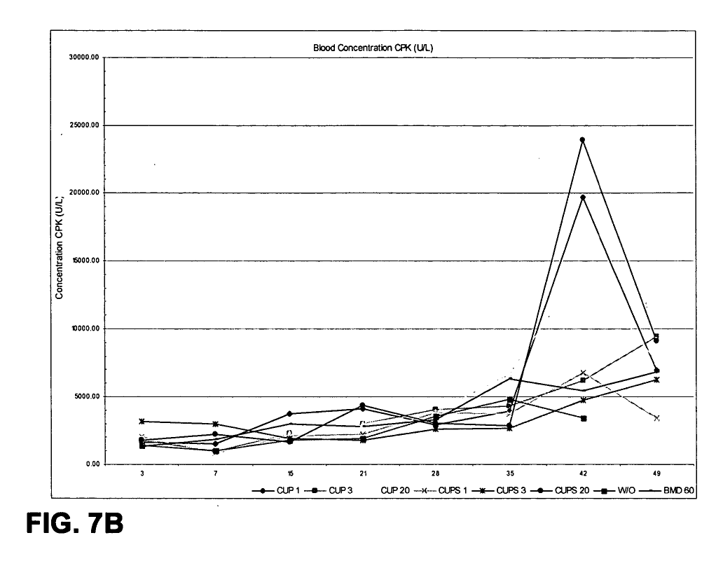

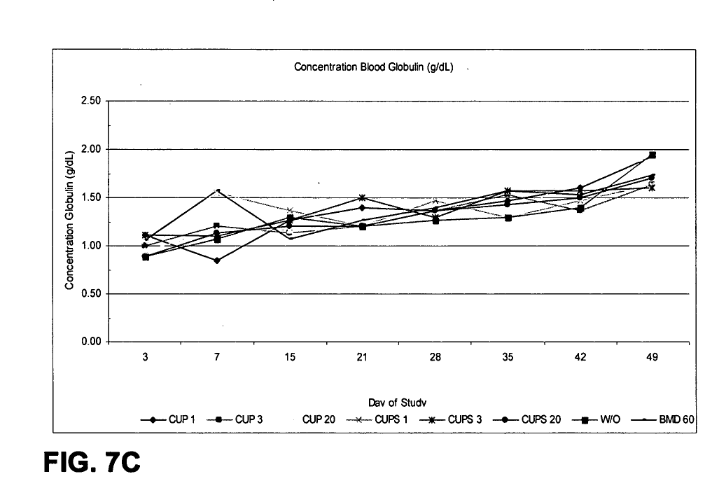

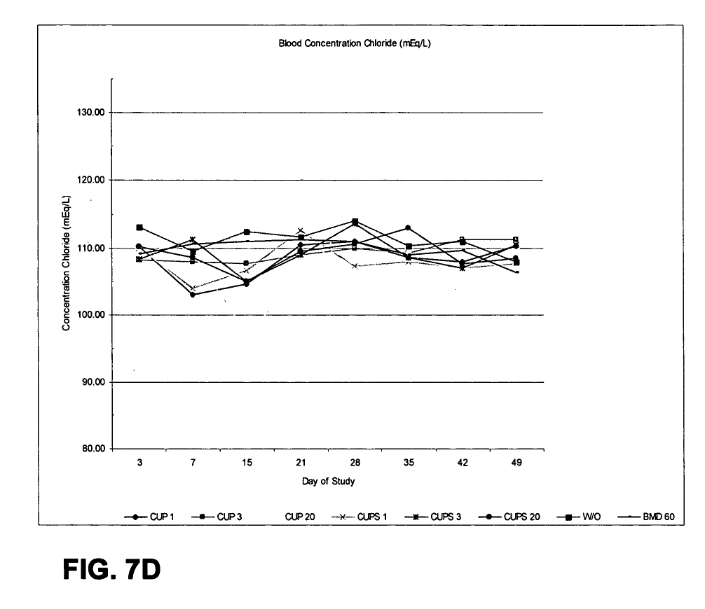

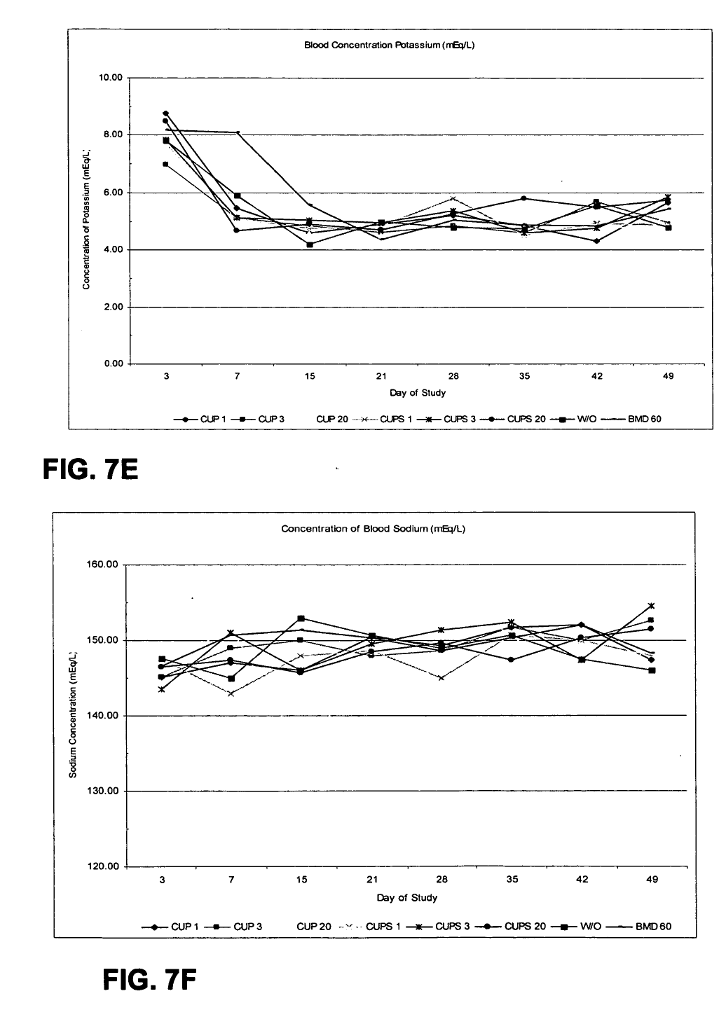

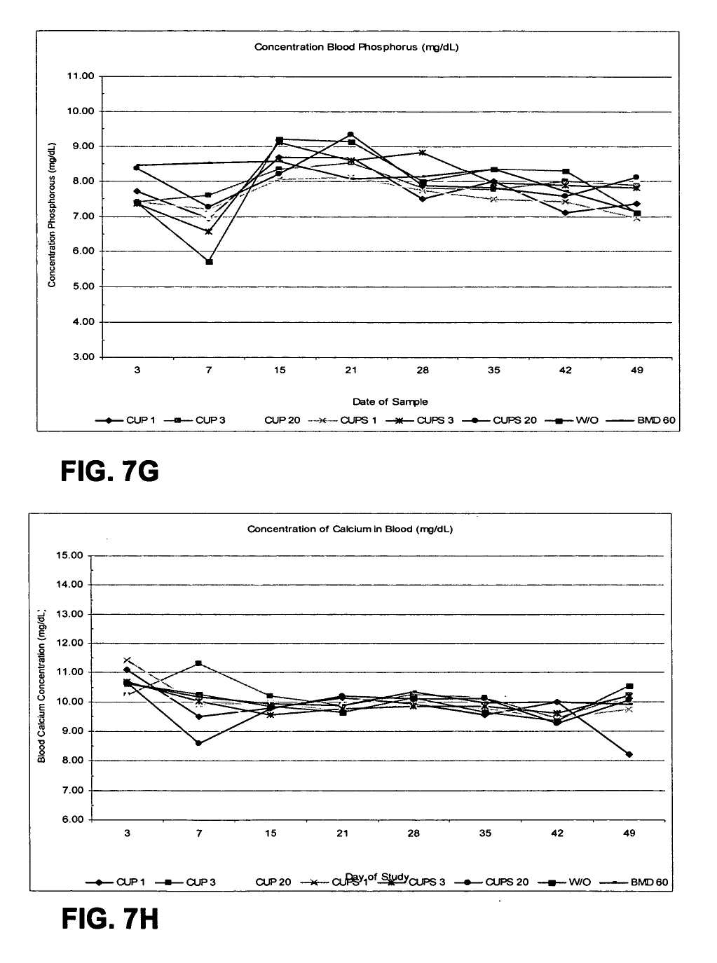

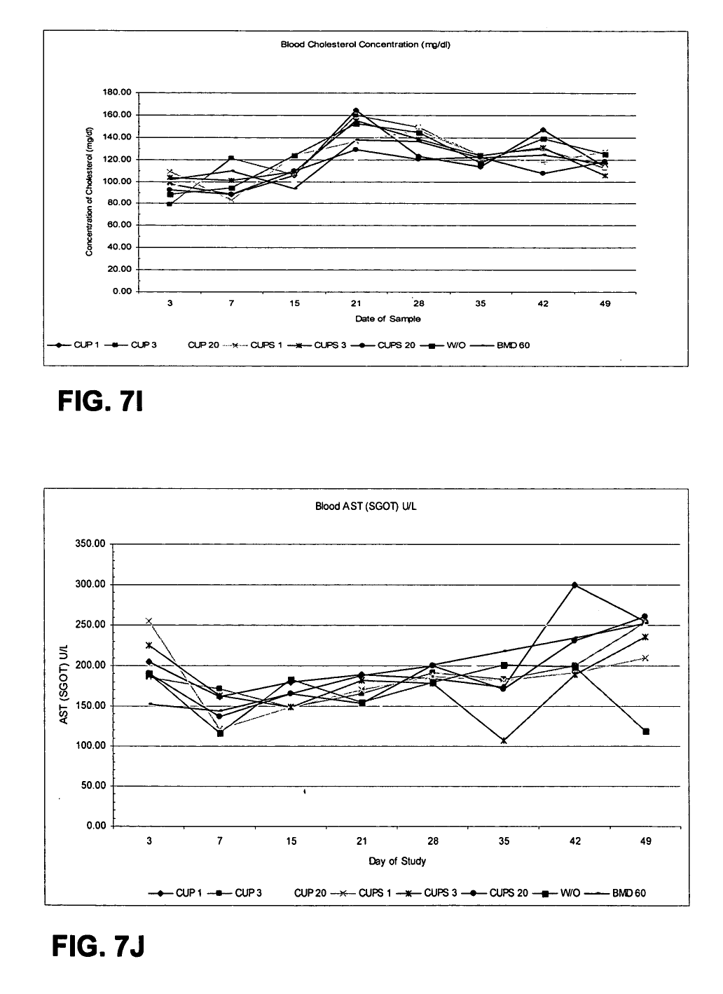

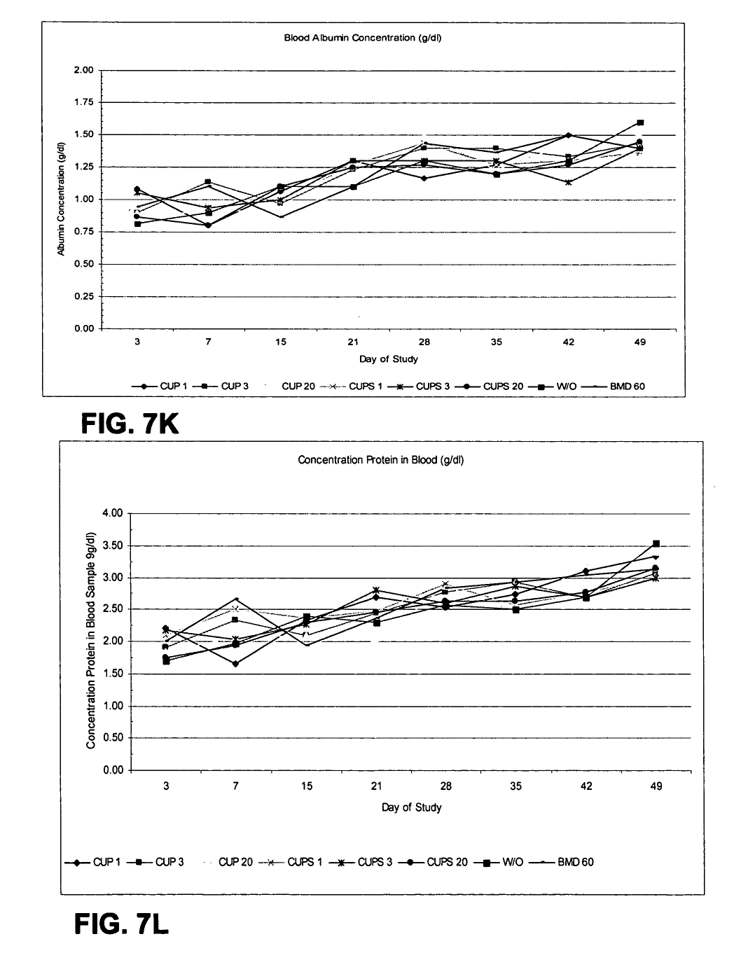

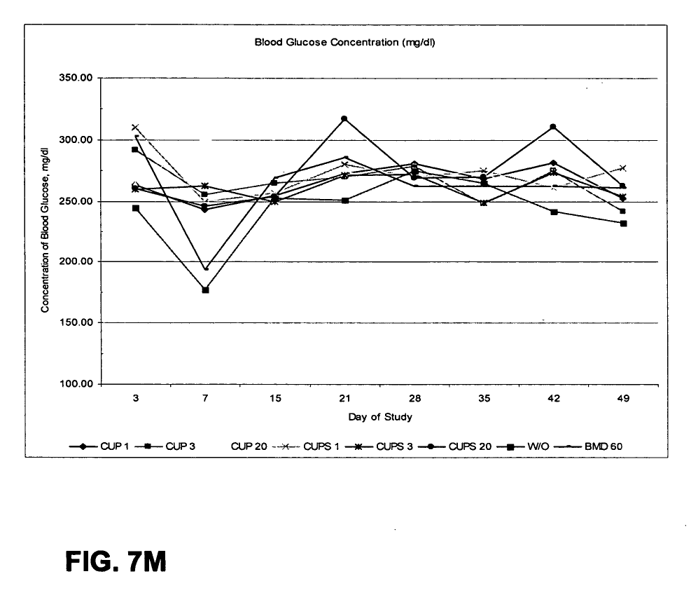

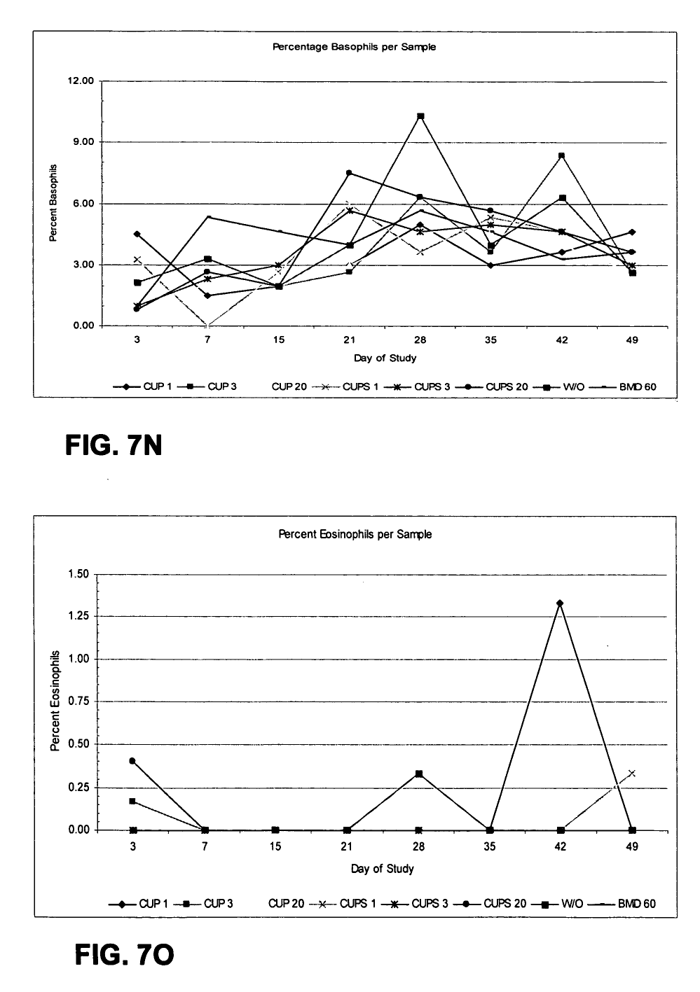

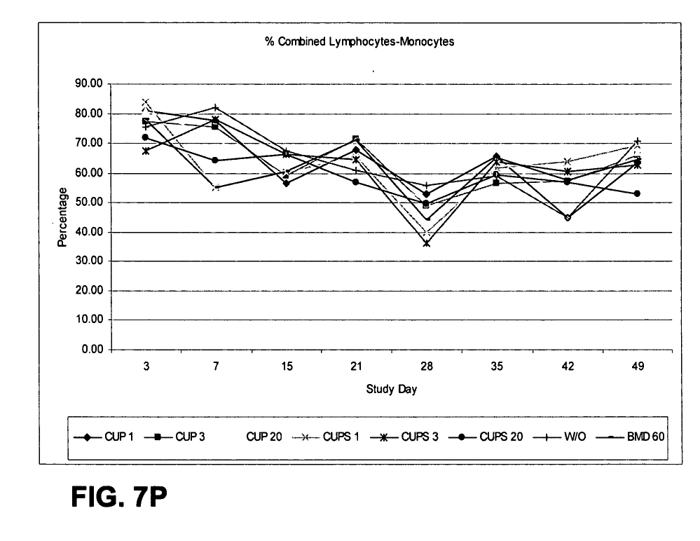

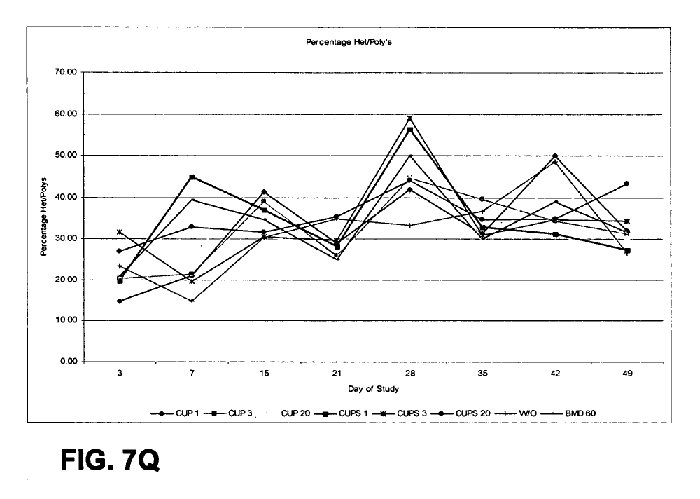

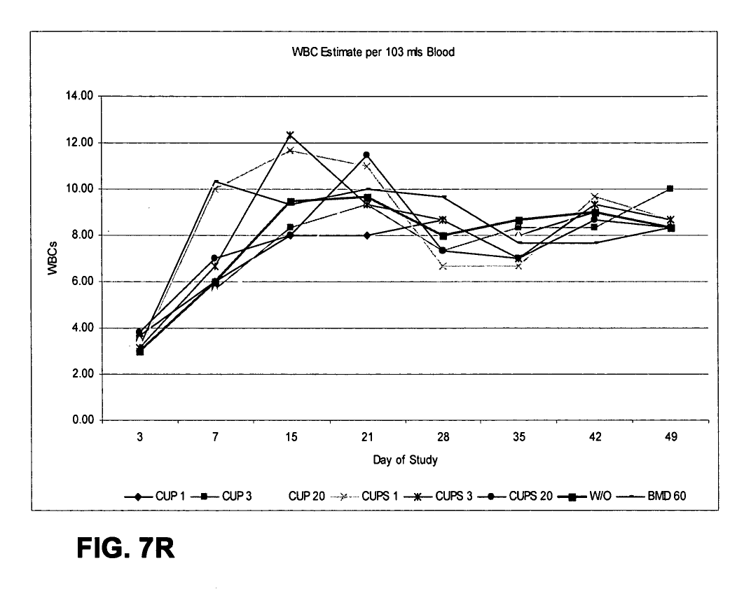

The blood chemistry data has been graphically summarized in FIGS. 7A through 7R.

TABLE 16
BLOOD CHEMISTRY RESULTS SUMMARIZED

| | | | | | | CUPS | | |
|---|---|---|---|---|---|---|---|---|
| Group | CUP 1 | CUP 3 | CUP 20 | CUPS 1 | CUPS 3 | 20 | W/O | BMD 60 |

14.1. Day 3 Chemistry

| | | | | | | | | | |
|---|---|---|---|---|---|---|---|---|---|
| Electrolyte Balance | | | | | | | | | |
| Ca | mg/dL | 11.10 | 10.22 | 10.23 | 11.43 | 10.70 | 10.67 | 10.62 | 10.62 |
| Cl | mEq/L | 110.17 | 108.17 | 110.50 | 110.00 | 108.33 | 110.17 | 113.17 | 109.20 |
| PHOS | mg/dL | 7.72 | 7.42 | 7.25 | 7.40 | 7.37 | 8.37 | 7.38 | 8.46 |
| K | mEq/L | 8.77 | 6.97 | 7.53 | 7.73 | 7.85 | 8.50 | 7.82 | 8.18 |
| Na | mEq/L | 145.17 | 145.17 | 146.33 | 147.50 | 143.50 | 146.50 | 147.67 | 146.60 |
| Glucose | | | | | | | | | |
| GLU | mg/dL | 263.17 | 291.50 | 263.67 | 309.75 | 260.00 | 260.83 | 244.67 | 302.80 |
| Liver Function, | | | | | | | | | |
| Hepatocellular | | | | | | | | | |
| AST | U/L | 204.67 | 185.67 | 181.83 | 254.50 | 225.50 | 190.17 | 191.00 | 152.00 |
| Kidney Function | | | | | | | | | |
| Uric Acid | mg/dL | 11.42 | 10.23 | 7.62 | 9.75 | 8.22 | 11.17 | 8.43 | 8.28 |
| Others | | | | | | | | | |
| Albumin | g/dL | 1.08 | 0.90 | 0.90 | 1.05 | 1.05 | 0.87 | 0.82 | 0.94 |
| Globulin | g/dL | 1.12 | 1.00 | 0.95 | 1.05 | 1.12 | 0.88 | 0.88 | 1.06 |
| Total Protein | g/dL | 2.20 | 1.90 | 1.85 | 2.10 | 2.17 | 1.75 | 1.70 | 2.00 |
| Cholesterol | mg/dL | 97.50 | 79.17 | 95.83 | 108.75 | 104.00 | 92.17 | 88.17 | 101.60 |
| CPK | U/L | 1643.33 | 1753.67 | 1419.17 | 2011.25 | 3178.83 | 1781.33 | 1374.00 | 1356.60 |

14.2. Day 7 Chemistry¹

| | | | | | | | | | |
|---|---|---|---|---|---|---|---|---|---|
| Electrolyte Balance | | | | | | | | | |
| Ca | mg/dL | 9.50^{ab} | 11.30^{a} | 10.10^{ab} | 9.90^{ab} | 10.03^{ab} | 8.60^{b} | 10.27^{ab} | 10.17^{ab} |
| Cl | mEq/L | 103.00 | 108.00 | 109.33 | 104.00 | 111.33 | 108.67 | 109.50 | 110.67 |
| PHOS | mg/dL | 6.95^{ab} | 7.60^{ab} | 7.00^{ab} | 7.20^{ab} | 6.57^{ab} | 7.27^{ab} | 5.73^{a} | 8.53^{b} |
| K | mEq/L | 5.45^{ab} | 5.13^{b} | 5.10^{b} | 5.10^{b} | 5.13^{b} | 4.67^{b} | 5.90^{ab} | 8.10^{a} |
| Na | mEq/L | 147.00 | 149.00 | 151.67 | 143.00 | 151.00 | 147.33 | 145.00 | 150.67 |
| Glucose | | | | | | | | | |
| GLU | mg/dL | 243.50 | 255.33 | 302.00 | 249.50 | 263.00 | 246.00 | 177.33 | 193.67 |
| Liver Function | | | | | | | | | |
| a) Hepatocellular | | | | | | | | | |
| AST | U/L | 162.50 | 171.33 | 126.67 | 121.50 | 163.33 | 136.67 | 117.00 | 144.33 |
| Kidney Function | | | | | | | | | |
| Uric Acid | mg/dL | 8.65 | 8.27 | 8.97 | 8.80 | 10.40 | 7.00 | 6.77 | 7.23 |
| Others | | | | | | | | | |
| Albumin | g/dL | 0.80 | 1.13 | 0.90 | 0.95 | 0.93 | 0.80 | 0.90 | 1.10 |
| Globulin | g/dL | 0.85 | 1.20 | 1.17 | 1.55 | 1.10 | 1.13 | 1.07 | 1.57 |
| Total Protein | g/dL | 1.65 | 2.33 | 2.07 | 2.50 | 2.03 | 1.93 | 1.97 | 2.67 |
| Cholesterol | mg/dL | 88.00 | 121.33 | 104.67 | 83.00 | 101.00 | 88.33 | 94.67 | 110.00 |
| CPK | U/L | 1548.50 | 944.67 | 1044.67 | 817.00 | 2994.67 | 2232.00 | 1039.67 | 1849.67 |

14.3. Day 15 Chemistry¹

| | | | | | | | | | |
|---|---|---|---|---|---|---|---|---|---|
| Electrolyte Balance | | | | | | | | | |
| Ca | mg/dL | 9.80 | 10.20 | 9.33 | 9.97 | 9.57 | 9.77 | 9.85 | 9.93 |
| Cl | mEq/L | 104.67 | 107.67 | 97.33 | 106.67 | 105.00 | 105.00 | 112.50 | 111.00 |
| PHOS | mg/dL | 8.70 | 8.33 | 8.20 | 8.03 | 9.10 | 8.20 | 9.20 | 8.57 |
| K | mEq/L | 4.60 | 4.83 | 4.60 | 4.77 | 5.03 | 4.90 | 4.20 | 5.57 |
| Na | mEq/L | 146.00 | 150.00 | 135.67 | 148.00 | 146.00 | 145.67 | 153.00 | 151.33 |
| Glucose | | | | | | | | | |
| GLU | mg/dL | 255.00 | 265.33 | 274.00 | 257.00 | 249.67 | 254.33 | 252.50 | 269.00 |
| Liver Function | | | | | | | | | |
| a) Hepatocellular | | | | | | | | | |
| AST | U/L | 179.33 | 149.33 | 151.33 | 148.33 | 149.33 | 165.67 | 183.00 | 166.00 |
| Kidney Function | | | | | | | | | |
| Uric Acid | mg/dL | 4.27^{ab} | 3.53^{b} | 51.33^{ab} | 6.97^{a} | 3.80^{b} | 3.63^{b} | 4.55^{ab} | 2.93^{b} |
| Others | | | | | | | | | |
| Albumin | g/dL | 1.07 | 0.97 | 0.93 | 1.00 | 1.00 | 1.10 | 1.10 | 0.87 |
| Globulin | g/dL | 1.27 | 1.13 | 1.17 | 1.37 | 1.27 | 1.20 | 1.30 | 1.07 |
| Total Protein | g/dL | 2.33 | 2.10 | 2.10 | 2.37 | 2.27 | 2.30 | 2.40 | 1.93 |
| Cholesterol | mg/dL | 105.67 | 106.33 | 100.67 | 124.00 | 109.00 | 109.67 | 124.00 | 93.67 |
| CPK | U/L | 3763.33 | 2269.00 | 2247.67 | 2064.67 | 1892.00 | 1615.33 | 1796.00 | 2965.33 |

14.4. Day 21 Chemistry

| | | | | | | | | | |
|---|---|---|---|---|---|---|---|---|---|
| Electrolyte Balance | | | | | | | | | |
| Ca | mg/dL | 10.15 | 9.87 | 9.93 | 9.67 | 9.75 | 10.20 | 9.63 | 9.87 |
| Cl | mEq/L | 110.50 | 109.00 | 105.00 | 112.67 | 109.00 | 109.50 | 111.67 | 111.33 |
| PHOS | mg/dL | 8.70 | 8.53 | 8.73 | 8.10 | 8.60 | 9.35 | 9.13 | 8.07 |
| K | mEq/L | 4.90 | 4.63 | 4.50 | 4.83 | 4.95 | 4.70 | 5.00 | 4.37 |
| Na | mEq/L | 150.50 | 148.00 | 146.33 | 148.67 | 149.50 | 148.50 | 150.67 | 150.33 |
| Glucose | | | | | | | | | |
| GLU | mg/dL | 273.00 | 270.00 | 275.33 | 280.33 | 271.50 | 317.00 | 251.00 | 286.00 |
| Liver Function | | | | | | | | | |
| a) Hepatocellular | | | | | | | | | |
| AST | U/L | 189.50 | 165.00 | 136.00 | 170.67 | 182.50 | 188.00 | 155.00 | 153.67 |
| Kidney Function | | | | | | | | | |
| Uric Acid | mg/dL | 4.35 | 3.30 | 4.17 | 3.90 | 4.50 | 3.85 | 3.67 | 4.67 |
| Others | | | | | | | | | |
| Albumin | g/dL | 1.30 | 1.23 | 0.83 | 1.27 | 1.30 | 1.25 | 1.10 | 1.10 |
| Globulin | g/dL | 1.40 | 1.20 | 1.17 | 1.20 | 1.50 | 1.20 | 1.20 | 1.27 |
| Total Protein | g/dL | 2.70 | 2.43 | 2.00 | 2.47 | 2.80 | 2.45 | 2.30 | 2.37 |
| Cholesterol | mg/dL | 164.50 | 160.00 | 120.00 | 136.33 | 155.00 | 129.00 | 152.33 | 138.00 |
| CPK | U/L | 4136.50 | 2951.33 | 2954.00 | 2212.33 | 1771.50 | 4375.00 | 1905.33 | 2771.67 |

14.5. Day 28 Chemistry¹

| | | | | | | | | | |
|---|---|---|---|---|---|---|---|---|---|
| Electrolyte Balance | | | | | | | | | |
| Ca | mg/dL | 9.93 | 10.27 | 10.50 | 9.93 | 9.83 | 10.10 | 10.13 | 10.33 |
| Cl | mEq/L | 111.00 | 110.00 | 115.33 | 107.33 | 113.67 | 110.67 | 114.00 | 111.00 |
| PHOS | mg/dL | 7.50 | 7.80 | 8.23 | 7.73 | 8.83 | 7.87 | 8.00 | 8.13 |
| K | mEq/L | 5.20 | 4.83 | 4.77 | 5.80 | 5.37 | 5.27 | 4.80 | 5.03 |
| Na | mEq/L | 149.33^{ab} | 148.67^{ab} | 156.33^{ab} | 145.00^{b} | 151.33^{ab} | 149.67^{a} | 149.00^{ab} | 148.67^{ab} |
| Glucose | | | | | | | | | |
| GLU | mg/dL | 280.67 | 278.67 | 260.00 | 270.00 | 272.00 | 269.33 | 275.00 | 262.67 |
| Liver Function | | | | | | | | | |
| a) Hepatocellular | | | | | | | | | |
| AST | U/L | 185.00 | 192.00 | 188.67 | 187.00 | 179.00 | 199.67 | 179.33 | 200.67 |
| Kidney Function | | | | | | | | | |
| Uric Acid | mg/dL | 4.07 | 3.57 | 4.27 | 3.63 | 2.70 | 3.63 | 3.43 | 4.20 |
| Others | | | | | | | | | |
| Albumin | g/dL | 1.17 | 1.40 | 1.50 | 1.43 | 1.30 | 1.27 | 1.30 | 1.43 |
| Globulin | g/dL | 1.37 | 1.37 | 1.37 | 1.47 | 1.30 | 1.37 | 1.27 | 1.40 |
| Total Protein | g/dL | 2.53 | 2.77 | 2.87 | 2.90 | 2.60 | 2.63 | 2.57 | 2.83 |
| Cholesterol | mg/dL | 123.00 | 149.33 | 150.67 | 146.67 | 138.67 | 120.33 | 144.67 | 136.33 |
| CPK | U/L | 2910.33 | 4030.00 | 3831.33 | 3820.67 | 2605.67 | 3063.33 | 3518.67 | 3262.00 |

14.6. Day 35 Chemistry

| | | | | | | | | | |
|---|---|---|---|---|---|---|---|---|---|
| Electrolyte Balance | | | | | | | | | |
| Ca | mg/dL | 9.57 | 10.13 | 10.10 | 9.77 | 9.83 | 10.10 | 9.63 | 9.97 |
| Cl | mEq/L | 108.67 | 109.33 | 109.67 | 108.00 | 108.67 | 113.00 | 110.33 | 109.00 |
| PHOS | mg/dL | 8.00 | 7.77 | 7.50 | 7.47 | 7.93 | 7.80 | 8.33 | 8.33 |
| K | mEq/L | 4.83 | 4.60 | 4.80 | 4.50 | 4.60 | 5.80 | 4.73 | 4.87 |
| Na | mEq/L | 151.67 | 151.67 | 150.67 | 150.67 | 152.33 | 147.33 | 150.67 | 150.33 |
| Glucose | | | | | | | | | |
| GLU | mg/dL | 268.33 | 248.33 | 273.00 | 275.00 | 248.67 | 270.00 | 265.33 | 263.00 |
| Liver Function | | | | | | | | | |
| a) Hepatocellular | | | | | | | | | |
| AST | U/L | 173.67 | 183.67 | 208.67 | 182.00 | 106.67 | 171.00 | 201.67 | 218.33 |
| Kidney Function | | | | | | | | | |
| Uric Acid | mg/dL | 3.07 | 2.80 | 4.13 | 3.67 | 2.33 | 2.37 | 3.10 | 3.20 |
| Others | | | | | | | | | |
| Albumin | g/dL | 1.27 | 1.40 | 1.23 | 1.27 | 1.30 | 1.20 | 1.20 | 1.37 |
| Globulin | g/dL | 1.47 | 1.53 | 1.23 | 1.30 | 1.57 | 1.43 | 1.30 | 1.57 |
| Total Protein | g/dL | 2.73 | 2.93 | 2.47 | 2.57 | 2.87 | 2.63 | 2.50 | 2.93 |
| Cholesterol | mg/dL | 113.67 | 124.00 | 122.33 | 124.00 | 123.67 | 122.33 | 117.33 | 121.67 |
| CPK | U/L | 3894.67 | 4297.67 | 6614.33 | 3718.00 | 2643.33 | 2875.00 | 4801.00 | 6324.00 |

14.7. Day 42 Chemistry¹

| | | | | | | | | | |
|---|---|---|---|---|---|---|---|---|---|
| Electrolyte Balance | | | | | | | | | |
| Ca | mg/dL | 10.00 | 9.43 | 10.33 | 9.43 | 9.60 | 9.27 | 9.35 | 10.00 |
| Cl | mEq/L | 108.00 | 111.33 | 103.00 | 107.00 | 107.00 | 107.67 | 111.00 | 109.67 |
| PHOS | mg/dL | 7.10 | 8.00 | 7.90 | 7.40 | 7.87 | 7.57 | 8.30 | 7.70 |
| K | mEq/L | 4.30 | 5.70 | 4.63 | 4.93 | 4.77 | 5.50 | 5.55 | 4.83 |
| Na | mEq/L | 152.00 | 150.00 | 143.67 | 150.00 | 147.33 | 150.33 | 147.50 | 152.00 |
| Glucose | | | | | | | | | |
| GLU | mg/dL | 282.00 | 275.33 | 253.67 | 261.00 | 273.33 | 310.67 | 242.00 | 262.67 |
| Liver Function | | | | | | | | | |
| a) Hepatocellular | | | | | | | | | |
| AST | U/L | 300.00 | 200.00 | 277.00 | 191.67 | 189.33 | 231.00 | 199.00 | 235.00 |
| Kidney Function | | | | | | | | | |
| Uric Acid | mg/dL | 3.40^{ab} | 3.10^{ab} | 3.23^{ab} | 3.70^{ab} | 2.27^{b} | 2.83^{ab} | 1.70^{b} | 4.83^{a} |
| Others | | | | | | | | | |
| Albumin | g/dL | 1.50 | 1.33 | 1.43 | 1.30 | 1.13 | 1.27 | 1.30 | 1.50 |
| Globulin | g/dL | 1.60 | 1.37 | 1.80 | 1.47 | 1.57 | 1.50 | 1.40 | 1.53 |
| Total Protein | g/dL | 3.10 | 2.70 | 3.23 | 2.77 | 2.70 | 2.77 | 2.70 | 3.03 |
| Cholesterol | mg/dL | 147.00 | 130.00 | 131.33 | 118.00 | 131.00 | 108.00 | 139.50 | 124.33 |
| CPK | U/L | 19710.00 | 6175.67 | 14607.00 | 6761.67 | 4774.67 | 23898.00 | 3430.50 | 5448.00 |

14.8. Day 49 Chemistry¹

| | | | | | | | | | |
|---|---|---|---|---|---|---|---|---|---|
| Electrolyte Balance | | | | | | | | | |
| Ca | mg/dL | 8.20 | 10.23 | 10.57 | 9.73 | 10.20 | 10.05 | 10.55 | 9.90 |
| Cl | mEq/L | 110.33 | 111.33 | 104.67 | 107.67 | 110.50 | 108.50 | 108.00 | 106.33 |
| PHOS | mg/dL | 7.37 | 7.87 | 8.03 | 6.93 | 7.80 | 8.10 | 7.10 | 7.13 |
| K | mEq/L | 5.67 | 4.97 | 5.10 | 4.87 | 5.85 | 5.75 | 4.80 | 5.43 |
| Na | mEq/L | 147.33 | 152.67 | 149.00 | 148.00 | 154.50 | 151.50 | 146.00 | 148.33 |
| Glucose | | | | | | | | | |
| GLU | mg/dL | 252.67^{ab} | 242.00^{b} | 267.67^{ab} | 277.00^{a} | 254.00^{ab} | 263.00^{ab} | 232.00^{b} | 261.67^{ab} |
| Liver Function | | | | | | | | | |
| a) Hepatocellular | | | | | | | | | |
| AST | U/L | 254.67 | 254.00 | 251.00 | 209.00 | 236.00 | 260.50 | 118.50 | 252.33 |
| Kidney Function | | | | | | | | | |
| Uric Acid | mg/dL | 3.00 | 3.97 | 3.87 | 4.00 | 3.90 | 3.75 | 3.30 | 3.63 |
| Others | | | | | | | | | |
| Albumin | g/dL | 1.40 | 1.43 | 1.50 | 1.37 | 1.40 | 1.45 | 1.60 | 1.40 |
| Globulin | g/dL | 1.93 | 1.63 | 1.90 | 1.63 | 1.60 | 1.70 | 1.95 | 1.73 |
| Total Protein | g/dL | 3.33 | 3.07 | 3.40 | 3.00 | 3.00 | 3.15 | 3.55 | 3.13 |
| Cholesterol | mg/dL | 114.00 | 112.33 | 114.00 | 127.33 | 105.50 | 118.50 | 125.00 | 116.67 |
| CPK | U/L | 6960.67 | 9460.00 | 7613.00 | 3388.50 | 6276.00 | 9140.00 | | 6845.50 |

¹Values with different letters are statistically (p < 0.05) different.

Hematology Statistical Results

There were no pair-wise comparison identified statistically significant differences between the W/O or BMD 60 and the other treatment compounds and dose groups throughout the measurement periods. While ANOVA identified statistically significant differences between treatment groups for Day 3 relative eosinophil count (p=0.03) and Day 3 log transformed relative basophil count (p=0.049), there was insufficient statistical power to pair-wise identify the existence of any significant difference between specific individual treatment groups. Hematology results are summarized in TABLE 17.

Hematology Clinical Results

All hematology ranges below are from adult chickens as reported in Avian Medicine: Principles and applications by Ritchie, Harrison and Harrison (1994). In my experience in sequential hematological evaluation of turkey specimens both percentages of cell types and white blood cell (WBC) counts vary significantly over the first 10 weeks of life. It is likely that the same occurs in broiler chickens and the best comparison population for the experimental data in this study are the W/O treatment group within the study. It is worth noting that no statistical differences between treatments were noted and therefore no treatment related effects are present.

Basophils typically account for 1.7 to 4.3% of WBCs in an adult chicken differential count and a mean value of 6.2% is reported in 10-day-old broiler chickens (Bartholomew et al., Biol. Trace Elem. Res. 62:7-16, 1998). The results in the current study are compatible with these ranges and interpreted to be normal.

Eosinophils typically account for 1.5 to 2.7% of WBCs in an adult chicken differential count and a mean value of 2.5% is reported in 10-day-old broiler chickens (Bartholomew et al., 1998). The results in the current study are compatible with these ranges and interpreted to be normal.

Heterophils typically account for 19.8 to 32.6% of WBCs in an adult chicken differential count and a mean value of 28.5% is reported in 10-day-old broiler chickens (Bartholomew et al., 1998). The results in the current study are compatible with these ranges and interpreted to be normal.

The lymphocyte and monocyte counts were combined for the following reasons: Birds have very similar lymphocyte and monocyte morphology that is differentiated by a number of arbitrary, frankly subjective, criteria. It is not uncommon to tolerate a misclassification rate of over 25% between lymphocytes and monocytes. In this study, the total number of lymphocytes and monocytes were within normal range. The number of monocyte count is low compared to the lymphocyte count. If either the lymphocytes or monocytes were to be individually elevated, the combined total number of lymphocytes and monocytes would reflect the elevation. With regard to birds, the most significant white blood cells in the leukogram interpretation are the heterophils and lymphocytes which were normal throughout the study. An increase in the monocyte count is generally an indication of chronic inflammation and this was not seen in the current study. When taken together, the overall good health of the birds, normal white blood cell count and the normal leukogram suggest that the combination of lymphocyte and monocyte counts do not impact the interpretation of the data.

The blood hematology results have been summarized graphically in FIGS. 7A through 7R.

TABLE 17
HEMATOLOGY RESULTS SUMMARIZED

| | | | | | | | | |
|---|---|---|---|---|---|---|---|---|
| Group | CUP 1 | CUP 3 | CUP 20 | CUPS 1 | CUPS 3 | CUPS 20 | W/O | BMD 60 |

15.1. Day 3 Hematology

| | | | | | | | | | |
|---|---|---|---|---|---|---|---|---|---|
| Red Blood Cells | | | | | | | | | |
| HCT | % | 30.60 | 28.67 | 27.00 | 30.67 | 27.80 | 27.00 | 27.83 | 26.20 |
| White Blood | | | | | | | | | |
| Cells | | | | | | | | | |
| BASO | X/uL | 193.33 | 78.33 | 48.33 | 127.50 | 45.00 | 16.00 | 50.00 | 34.00 |
| EOS | X/uL | 0.00 | 6.67 | 0.00 | 0.00 | 0.00 | 16.00 | 0.00 | 0.00 |
| LYMP + MONO | X/uL | 2956.67 | 2595.00 | 2828.33 | 2590.00 | 2060.00 | 2950.00 | 2560.00 | 2558.00 |
| NEUT | X/uL | 516.67 | 820.00 | 623.33 | 782.50 | 1061.67 | 818.00 | 398.33 | 608.00 |
| WBC | ×10\hat{ }3/uL | 3.67 | 3.50 | 3.50 | 3.00 | 3.17 | 3.80 | 3.00 | 3.20 |
| Others | | | | | | | | | |
| Het/Poly | % | 14.67 | 20.33 | 17.67 | 19.75 | 31.67 | 27.00 | 23.33 | 20.80 |

15.2. Day 7 Hematology

| | | | | | | | | | |
|---|---|---|---|---|---|---|---|---|---|
| Red Blood Cells | | | | | | | | | |
| HCT | % | N/A | N/A | N/A | N/A | N/A | N/A | N/A | N/A |
| White Blood | | | | | | | | | |
| Cells | | | | | | | | | |
| BASO | X/uL | 85.00 | 193.33 | 113.33 | 0.00 | 146.67 | 180.00 | 213.33 | 396.67 |
| EOS | X/uL | 0.00 | 0.00 | 0.00 | 0.00 | 0.00 | 0.00 | 0.00 | 0.00 |
| LYMP + MONO | X/uL | 4665.00 | 4253.33 | 4050.00 | 5500.00 | 5273.33 | 4513.33 | 4870.00 | 5553.33 |
| NEUT | X/uL | 1250.00 | 1220.00 | 1503.33 | 4500.00 | 1246.67 | 2306.67 | 916.67 | 4383.33 |
| WBC | ×10\hat{ }3/uL | 6.00 | 5.67 | 5.67 | 10.00 | 6.67 | 7.00 | 6.00 | 10.33 |
| Others | | | | | | | | | |
| Het/Poly | % | 21.00 | 21.33 | 25.67 | 45.00 | 19.67 | 33.00 | 14.67 | 39.33 |

15.3. Day 15 Hematology

| | | | | | | | | | |
|---|---|---|---|---|---|---|---|---|---|
| Red Blood Cells | | | | | | | | | |
| HCT | % | 27.33 | 31.00 | 22.00 | 32.00 | 29.00 | 24.33 | 32.50 | 27.33 |
| White Blood | | | | | | | | | |
| Cells | | | | | | | | | |
| BASO | X/uL | 146.67 | 153.33 | 113.33 | 266.67 | 406.67 | 180.00 | 175.00 | 350.00 |
| EOS | X/uL | 0.00 | 0.00 | 0.00 | 0.00 | 0.00 | 0.00 | 0.00 | 0.00 |
| LYMP + MONO | X/uL | 4346.67 | 4796.67 | 3710.00 | 6963.33 | 8000.00 | 5213.33 | 6405.00 | 5753.33 |
| NEUT | X/uL | 3506.67 | 3383.33 | 2843.33 | 4436.67 | 3926.67 | 2606.67 | 2920.00 | 3230.00 |
| WBC | ×10\hat{ }3/uL | 8.00 | 8.33 | 6.67 | 11.67 | 12.33 | 8.00 | 9.50 | 9.33 |
| Others | | | | | | | | | |
| Het/Poly | % | 41.33 | 39.00 | 45.33 | 37.00 | 30.67 | 31.67 | 30.50 | 34.67 |

5.4. Day 21 Hematology

| | | | | | | | | | |
|---|---|---|---|---|---|---|---|---|---|
| Red Blood Cells | | | | | | | | | |
| HCT | % | 30.00 | 30.33 | 31.00 | 30.33 | 31.00 | 31.50 | 31.33 | 28.67 |
| White Blood | | | | | | | | | |
| Cells | | | | | | | | | |
| BASO | X/uL | 240.00 | 250.00 | 306.67 | 630.00 | 523.33 | 850.00 | 415.00 | 400.00 |
| EOS | X/uL | 0.00 | 0.00 | 0.00 | 0.00 | 0.00 | 0.00 | 0.00 | 0.00 |
| LYMP + MONO | X/uL | 5440.00 | 6640.00 | 6746.67 | 7243.33 | 6016.67 | 6530.00 | 5886.67 | 7113.33 |
| NEUT | X/uL | 2320.00 | 2443.33 | 3280.00 | 3126.67 | 2793.33 | 4120.00 | 3396.67 | 2486.67 |
| WBC | ×10\hat{ }3/uL | 8.00 | 9.33 | 10.33 | 11.00 | 9.33 | 11.50 | 9.67 | 10.00 |
| Others | | | | | | | | | |
| Het/Poly | % | 29.00 | 26.00 | 32.67 | 28.33 | 29.67 | 35.50 | 35.00 | 25.00 |

15.5. Day 28 Hematology

| | | | | | | | | | |
|---|---|---|---|---|---|---|---|---|---|
| Red Blood Cells | | | | | | | | | |
| HCT | % | 29.33 | 30.00 | 33.67 | 33.00 | 29.67 | 27.33 | 29.33 | 29.00 |
| White Blood | | | | | | | | | |
| Cells | | | | | | | | | |
| BASO | X/uL | 446.67 | 560.00 | 410.00 | 260.00 | 450.00 | 393.33 | 843.33 | 570.00 |
| EOS | X/uL | 0.00 | 0.00 | 0.00 | 0.00 | 0.00 | 0.00 | 30.00 | 0.00 |
| LYMP + MONO | X/uL | 4546.67 | 3323.33 | 4413.33 | 2300.00 | 3216.67 | 3386.67 | 4350.00 | 4260.00 |
| NEUT | X/uL | 3673.33 | 3450.00 | 3843.33 | 4106.67 | 5000.00 | 3553.33 | 2776.67 | 4836.67 |
| WBC | ×10\hat{ }3/uL | 8.67 | 7.33 | 8.67 | 6.67 | 8.67 | 7.33 | 8.00 | 9.67 |
| Others | | | | | | | | | |
| Het/Poly | % | 42.00 | 44.67 | 45.00 | 56.33 | 59.00 | 44.00 | 33.33 | 50.00 |

15.6. Day 35 Hematology

| | | | | | | | | | |
|---|---|---|---|---|---|---|---|---|---|
| Red Blood Cells | | | | | | | | | |
| HCT | % | 29.67 | 31.67 | 28.67 | 29.67 | 29.00 | 25.50 | 28.33 | 29.50 |
| White Blood | | | | | | | | | |
| Cells | | | | | | | | | |
| BASO | X/uL | 253.33 | 310.00 | 350.00 | 386.67 | 423.33 | 393.33 | 353.33 | 360.00 |
| EOS | X/uL | 0.00 | 0.00 | 0.00 | 0.00 | 0.00 | 0.00 | 0.00 | 0.00 |
| LYMP + MONO | X/uL | 5046.67 | 4876.67 | 5230.00 | 4026.67 | 4366.67 | 4323.33 | 5126.67 | 5036.67 |
| NEUT | X/uL | 2700.00 | 3146.67 | 2420.00 | 2253.33 | 2210.00 | 2283.33 | 3186.67 | 2270.00 |
| WBC | ×10\hat{ }3/uL | 8.00 | 8.33 | 8.00 | 6.67 | 7.00 | 7.00 | 8.67 | 7.67 |
| Others | | | | | | | | | |
| Het/Poly | % | 31.33 | 39.67 | 31.00 | 33.00 | 31.00 | 34.67 | 36.67 | 30.00 |

15.7. Day 42 Hematology

| | | | | | | | | | |
|---|---|---|---|---|---|---|---|---|---|
| Red Blood Cells | | | | | | | | | |
| HCT | % | 27.67 | 27.50 | 34.67 | 30.33 | 29.33 | 28.67 | 26.67 | 29.33 |
| White Blood | | | | | | | | | |
| Cells | | | | | | | | | |
| BASO | X/uL | 353.33 | 710.00 | 360.00 | 450.00 | 466.67 | 406.67 | 586.67 | 233.33 |
| EOS | X/uL | 106.67 | 0.00 | 0.00 | 0.00 | 0.00 | 0.00 | 0.00 | 0.00 |
| LYMP + MONO | X/uL | 3913.33 | 4816.67 | 4960.00 | 6200.00 | 5760.00 | 4980.00 | 4033.33 | 4396.67 |
| NEUT | X/uL | 4626.67 | 2806.67 | 4013.33 | 3016.67 | 3106.67 | 3013.33 | 4380.00 | 3036.67 |
| WBC | ×10\hat{ }3/uL | 9.00 | 8.33 | 9.33 | 9.67 | 9.33 | 8.67 | 9.00 | 7.67 |
| Others | | | | | | | | | |
| Het/Poly | % | 50.00 | 34.33 | 43.00 | 31.33 | 34.67 | 35.00 | 48.67 | 39.00 |

15.8. Day 49 Hematology

| | | | | | | | | | |
|---|---|---|---|---|---|---|---|---|---|
| Red Blood Cells | | | | | | | | | |
| HCT | % | 30.00 | 29.67 | 31.33 | 29.00 | 30.67 | 30.33 | 27.00 | 30.00 |
| White Blood | | | | | | | | | |
| Cells | | | | | | | | | |
| BASO | X/uL | 370.00 | 263.33 | 236.67 | 213.33 | 243.33 | 296.67 | 226.67 | 336.67 |
| EOS | X/uL | 0.00 | 0.00 | 0.00 | 40.00 | 0.00 | 0.00 | 0.00 | 0.00 |
| LYMP + MONO | X/uL | 5266.67 | 6606.67 | 5846.67 | 5766.67 | 5510.00 | 4483.33 | 5873.33 | 5220.00 |
| NEUT | X/uL | 2696.67 | 3130.00 | 2583.33 | 2646.67 | 2913.33 | 3553.33 | 2233.33 | 2776.67 |
| WBC | ×10\hat{ }3/uL | 8.33 | 10.00 | 8.67 | 8.67 | 8.67 | 8.33 | 8.33 | 8.33 |
| Others | | | | | | | | | |
| Het/Poly | % | 32.00 | 31.33 | 30.33 | 27.33 | 34.33 | 43.33 | 26.67 | 31.67 |

Acute Phase Protein Results

The alpha-1-acid glycoprotein evaluated in this study was used to evaluate the acute phase protein response in the broiler chickens. Since these chickens were in adequate commercial broiler conditions with no significant disease problems it is anticipated that only isolated broilers which have contracted a disease condition (such as colibacillosis) would have increased alpha-1-acid glycoprotein levels.

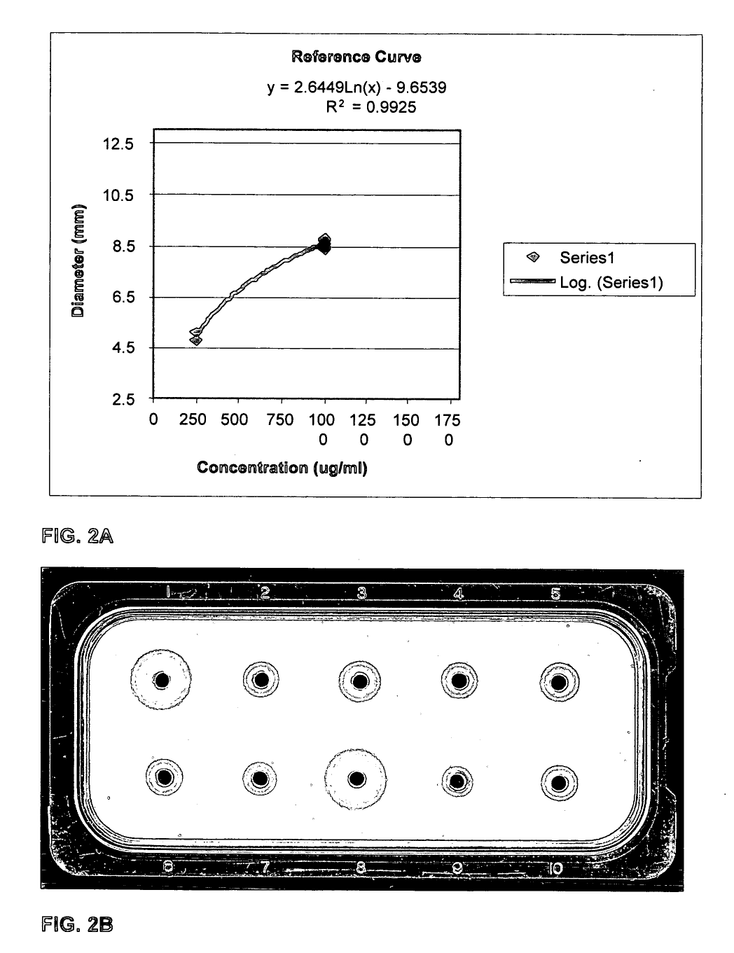

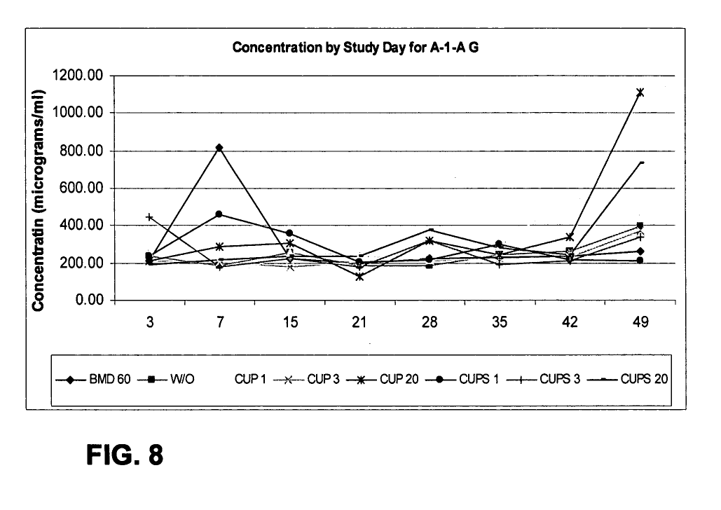

Each test sample thought to contain Chicken AGP was placed in an individual test well. As the sample diffused radially from the well into the agar gel plate, a specific precipitin reaction occurred between Chicken AGP and the specific antiserum to Chicken AGP incorporated in the gel. A visible precipitin ring was formed. Since the area within this ring was directly proportional to the concentration of AGP in the test sample, measurement of the ring's diameter allowed calculation of that AGP concentration, as compared to the two known standards, Solutions A and B. FIG. 2A is a graph summarizing the standard curve used to determine the concentration based on ring size. Using the equation: y=2.6449Ln(x)−9.6539 where y is the ring diameter, x then equals the concentration. The equation is rearranged to obtain the equation: e^{(y+9.6539)/2.6449} which yields the concentration of the alpha-1-acid glycoprotein. FIG. 2B shows an example digital image of the radial diffusion gels displaying typical results for an Acute Phase Protein assay. The alpha-1-acid glycoprotein levels observed in this study confirm this reasoning. The results are summarized in Table 18 and graphically presented in FIG. 8.

TABLE 18
ACUTE PHASE PROTEIN SUMMARIZATION
Concentration (μg/ml) alpha-1-acid glycoprotein

| Tx | Day 3 | Day 7 | Day 15 | Day 21 | Day 28 | Day 35 | Day 42 | Day 49 |
|---|---|---|---|---|---|---|---|---|
| W/O | 233.59 | 182.00 | 257.8333 | 187.44 | 186.38 | 244.32 | 259.44 | 397.01 |
| BMD 60 | 208.53 | 816.60 | 225.3311 | 196.04 | 225.70 | 231.25 | 238.61 | 260.14 |
| CUP 1 | 255.72 | 113.86 | 258.0617 | 236.14 | 215.89 | 287.14 | 563.08 | 449.24 |
| CUP 3 | 208.68 | 203.08 | 181.8196 | 201.40 | 208.29 | 222.92 | 232.53 | 370.68 |
| CUP 20 | 213.71 | 287.31 | 304.0475 | 129.01 | 321.74 | 241.15 | 341.29 | 1113.18 |
| CUPS 1 | 234.42 | 460.26 | 358.0647 | 207.30 | 217.45 | 297.67 | 216.35 | 209.85 |
| CUPS 3 | 444.16 | 176.02 | 225.6315 | 178.52 | 320.09 | 190.44 | 211.96 | 337.71 |
| CUPS 20 | 190.92 | 218.22 | 236.3457 | 234.68 | 375.69 | 281.86 | 241.13 | 734.57 |

Bacteriology Results

On Study Day 49 gut samples were taken from 3 birds from each pen. These results were sent to Antech Diagnostics for determination of Salmonella spp. and Campylobacter spp. The results returned all negative for all birds. These results were determined to be inconclusive.

### Conclusion

The goal of this pilot study was to evaluate the efficacy of two (2) products in broiler chickens as a growth promoting agents. These two (2) products were compared with an antibiotic that is commonly used in the United States in subtherapeutic levels as a growth promoting agent. For this pilot study the two (2) products were added to a basal diet at three (3) different dose levels (1, 3, and 20 mg active test article/kg body weight gain) for a total of 6 different diet treatments (3 levels x 2 articles=6 rations). The antibiotic was added at 60 g active ingredient per ton (˜909 kg) of feed. For this study the basal ration, free of any growth promoting agent, was included in the study for a total of 8 different diets.

On Day seven (7) of the study all of the product rations out performed both the BMD 60 group and the W/O group in terms of body weight gain (Table 4) while maintaining a feed intake and feed efficiency similar to the BMD 60 group. On Study Day fifteen (15) the product groups continued to perform as well or slightly better than the BMD 60 and all doing better than the W/O group in terms of body weight gain. All of the product groups showed an improvement in feed efficiency compared to both the BMD 60 group and the W/O group. The CUPS 20 group statistically (p<0.05) out performed the W/O for this phase on both body weight gain and feed efficiency, Tables 2 and 4 respectively. All of the product groups continued to perform as well as or better than the BMD 60 group throughout the rest of the trial for body weight gain and feed efficiency.

Though this was only a pilot study the data collected here for the body weight gain and feed efficiency consistently showed a positive trend for both products performance when compared to the performance of either the BMD 60 group or the W/O group. Because this study was designed to be a pilot study with a small number of animals the ability of our statistics to pick up differences was limited. Regardless of statistical power, this study readily showed that these two (2) products consistently performed as well as the antibiotic group as a growth promoter at all stages of the production cycle while not compromising the feed efficiency of the bird. It is clear that these products have some form of growth promoting effect in broilers that is as efficacious as a well-recognized antibiotic (BMD 60).

As an overall conclusion on the clinical pathology, hematology and acute phase protein data it appears that the broiler chickens in this study were healthy based on a very broad range of physiological parameters and can be concluded that neither of these products had any negative effects on the overall health of the bird.

Those skilled in the art will appreciate that the foregoing description teaches by way of example, and not by limitation. Accordingly, what is shown and described should be construed in a manner that is consistent with the scope and spirit of the invention

### REFERENCES

The following documents are incorporated by reference to the same extent as though fully replicated herein.
¹ Choct M C (2001) Alternatives To In-Feed Antibiotics In Monogastric Animal Industry. ASA Technical Bulletin Vol. AN30-2001 p. 1-6
² Mathew A (2002) Seeking Alternatives to Growth Promoting Antibiotics. Depart. Of Animal Science, The Uni. Of Tennessee, Knoxyille Tenn., USA
³ Turner J L, Pas, Dritz S S, and Minton J E (?) review: Alternatives to Conventional Antimicrobials in Swine Diets. The Professional Animal Scientist 17. p. 217-226
⁴ Mitchener B (1999) EU Moves Toward a Total Ban of Antibiotics in Animal Feed. Wall Street Journal, Jul. 28, 1999
⁵ Newman K (1994). Mannan-oligosaccharides: Natural Polymers with significant impact on the gastrointestinal microflora and the immune system. In: Lyons TP and J, KA (ed.) Biotechnology in the Feed Industry. Nottingham University Press, Nicholasville, Ky., p. 167-180
⁶ Waldroup P W, Oviedo-Rondon EO, fritss CA (2003) Comparison of Bio-Mos and Antibiotic Feeding Programs in Broiler Diets Containing Copper Sulfate. International Journal of Poultry Science 2 p 28-31
⁷ Fritts C A and Walroup P W (2003) Evaluation of Bio-Mos® Mannan Oligosaccharide as a replacement for Growth Promoting Antibiotics in Diets for Turkeys. International Journal of Poultry Science 2 p 19-22
⁸ Parks C W, Grimes J L, Ferket P R, and Fairchild A S (2001). The Effect of Mannanoligosaccharides, Bambermycins, and Virginiamycin on Performance of Large White Male Market Turkeys. Poultry Science 80 p 718-723
⁹ LeMieux F M, Southern L L, and Bidner T D (2003) Effect of mannan oligosaccharides on growth performance of weanling pigs. J. Animal Sci. 81 p 2482-2487
¹⁰ Davis M E, Maxwell C V, Brown D C, de Rodas B Z, Johnson Z B, Kegley E B, Hellwig D H, and Dvorak R A (2002) Effect of dietary mannan oligosaccharides and(or) pharmacological additions of copper sulfate on growth performance and immunocompetence of weanling and growing/finishing pigs. J. Animal Sci. 80 (2887-2894)
¹¹ Davies M E, Maxwell CV, Erf G F, Brown DC, and Wistuba T J (2004). Dietary supplementation with phosphorylated mannans improves growth response and modulates immune function of weanling pigs. J. Animal Sci. 82 p 1882-1891
¹² Franklin S T, Newman M C, Newman K E, and Meek K I (2005) Immune Paramteres of Dry Cows Fed Mannan Oligosaccharide and Subsequent Transfer of Immunity to Calves. J. Dairy Sci. 88 p 766-775
¹³ Buddington K K, Donahoo J B, Buddington R K (2002) Dietary Oligofructose and Inulin Protect Mice from Enteric and Systemic Pathogens and Tunor Inducers. P 472-477
¹⁴ Swanson K S, Grieshop C M, Flickinger E A, Bauer L L, Healy H P, Dawson K A, Merchen N R, and Fahey G C (2002) Supplemental Fructooligosaccharides and Mannanoligosaccharides Influence Immune Function, Ileal and Total Tract Nutrient Digestibilities, Microbal Populations and Concentrations of Protein Catabolites in the Large Bowel of Dogs. Nutritional Immunology p 980-989
¹⁵ Fernandez F, Hinton M, and Van Gils B (2002) Dietary mannan-oligosaccharides and their effect on chicken caecal microflora in relation to Salmonella Enteritidis colonization. Avian Pathology 31 p 49-58.
¹⁶ Allen V M, Fernandez F, and Hinton M H (1997). Evaluation of the influence of supplementing the diet with mannose or palm kernel meal on salmonella colonization in poultry. British Poultry Science 38 p 485-488
¹⁷ Tizard R I, Carpenter R H, McAnalley B H, and Kemp MC (1989) The biological activities of mannans and related complex carbohydrates. Mol. Biother. 1 p 290-296
¹⁸ Krizkova L, Durackova Z, Sandula J, Sasinkova V, and Krajcovic J (2001) Antioxidative and antimutagenic activity of yeast cell wall mannans in vitro. Mutation Research 497 p. 213-222
¹⁹ Djeraba A and Quere P (2000) In vivo macrophage activation in chickens with Acemannan, a complex carbohydrate extracted from Aloe vera. International Journal of Immunopharmacology 22 p. 365-372
²⁰ Olivella J G, and Torrus E F (1997) Study of the immunostimulating effect of glycophosphopeptical (AM3) in mice. FEMS Immunology and Medical Microbiology 18 p. 87-89
²¹ Villarrubia V G, Moreno Koch M C, Calvo C, Gonzalez S, and Alvarez-Mon M (1997) The immunosenescent phenotype in mice and humans can be defined by alterations in the natural immunity reversal by immunomodulation with oral AM3. Immunopharmacology and Immunotoxicology 19 p 53-74
²² Prieto A, Reyes E, Bernstein E D, Martinez B, et al (2001) Defective Natural Killer and Phagocyctic Activities in Chronic Obstructive Pulmonary Disease Are Restored by Glycophosphopeptical (Immunoferon). Am J Respir Crit Care Med Vol. 163 p 1578-1583
²³ Brieva A., Guerrero A, Pivel J P. (2002) Immunoferon, a glycoconjugate of natural origin, regulates the liver response to inflammation and inhibits TNF-α production by an HPA axis-dependent mechanism. Internation Immunopathology Vol. 275 p
²⁴ Brieva A, Guerrero A, Alonso-Lebrero J L, Pivel J P (2001). Immunoferon, a glycoconjugate of natural origin, inhibits LPS-induced TNF-α production and inflammatory responses. International Immunopharmacology Vol 1 p 1979-1987
²⁵ Johnson R W, (1997). Inhibition of Growth by Pro-Inflammatory Cytokines: An intergrated View. J Anim Sci Vol 75 p 1244-1255
²⁶ Podzorski R P, Gray G R, and Nelson R D (1990) Different Effects of Native Candida albicans Mannan and Mannan-Derived Oligosaccharides on Antigen-Stimulated Lymphoproliferation In Vitro. The Journal of Immunology Vol. 144 P 707-716.

1. A method of supplementing a poultry diet, the method comprising the steps of:
mixing poultry feed with a phosphorylated glucomannan polysaccharide in an effective amount to benefit poultry production, in order to provide a mixed poultry feed.

2. The method of claim 1, further comprising a step of feeding the mixed poultry feed to poultry to obtain a poultry production benefit from use of the phosphorylated glucomannan polysaccharide.

3. The method of claim 2, wherein the poultry production benefit includes at least one benefit selected from the group consisting of: increased poultry weight gain, increased relative quantities of the beneficial bacteria in the poultry, decreased relative quantities of malicious bacteria in the poultry, increased uptake of beneficial minerals, nutrients and vitamins; increased uptake of zinc and copper, improved overall general health of the poultry, and combinations thereof.

4. The method of claim 3, wherein the poultry production benefit includes increased muscle mass.

5. The method of claim 1, wherein the phosphorylated glucomannan contains a repeating polysaccharide subunit that is repeated approximately n times of 1-6 and 1-2 linkages between and within mannose and glucose residues at a ratio of 12:1 mannose:glucose, were n ranges from 10 to 40.

6. The method of claim 5, wherein n ranges from 10 to 20.

7. The method of claim 5, wherein n ranges from 20 to 30.

8. The method of claim 5, wherein n ranges from 30 to 40.

9. The method of claim 5, wherein n ranges from 20 to 40.

10. The method of claim 5, wherein the phosphorylated glucomannan is complexed with a protein.

11. The method of claim 10, wherein the phosphorylated glucomannan and protein are combined with a matrix or carrier.

12. The method of claim 11, wherein the matrix or carrier is inorganic.

13. The method of claim 5, wherein the phosphorylated glucomannan is combined with a matrix or carrier.

14. The method of claim 11, wherein the matrix or carrier is inorganic.

15. The method of claim 1, wherein the poultry production benefit is at least selected from the group consisting of reducing the subtherapeutic dose of antibiotic needed to accelerate weight gain; eliminating subtherapeutic doses of antibiotic in the starting and growing of feeder poultry, and eliminating subtherapeutic doses of antibiotics in the starting and growing of poultry.

16. The method of claim 1, wherein the step of mixing includes combining ingredients to form a liquid, gel, or colloid.

17. The method of claim 1, wherein the step of mixing includes combining ingredients to form a solid.

18. The method of claim 1 wherein the step of mixing includes combining ingredients that include a predetermined formulation of nutrients that target a specific stage of poultry development.

19. In a poultry feed, the improvement comprising:
a phosphorylated glucomannan polysaccharide mixed with the poultry feed in an effective amount to benefit poultry production.

20. The poultry feed of claim 19, wherein the poultry feed is formulated for optimal benefit at a nursery stage of poultry development.

21. The poultry feed of claim 19, wherein the poultry feed is formulated for optimal benefit at a feeder stage of poultry development.

22. The poultry feed of claim 19, wherein the poultry feed is formulated for optimal benefit of a maintenance stage of poultry development.

23. The poultry feed of claim 19, wherein the effective amount includes an amount ranging from 1 mg to 5 mg per kg of body weight based upon a targeted intake of food for the poultry.
